(19) United States
(12) Patent Application Publication
(10) Pub. No.: US 2014/0137278 A1
(43) Pub. Date: May 15, 2014

(54) METHODS AND COMPOSITIONS FOR PRODUCING NEMATODE RESISTANT COTTON PLANTS

(71) Applicant: Monsanto Technology LLC, St. Louis, MO (US)

(72) Inventors: Muhammad Bhatti, Ballwin, MO (US); Roy G. Cantrell, St. Peters, MO (US); Seungho Cho, Chesterfield, MO (US); Issa Coulibaly, Saint Peters, MO (US); Bill L. Hendrix, West Sacramento, CA (US); Don Lee Keim, Leland, MS (US); Kunsheng Wu, Ballwin, MO (US); Jinhua Xiao, Ballwin, MO (US)

(73) Assignee: Monsanto Technology LLC, St. Louis, MO (US)

(21) Appl. No.: 14/044,647

(22) Filed: Oct. 2, 2013

Related U.S. Application Data
(60) Provisional application No. 61/709,049, filed on Oct. 2, 2012.

(51) Int. Cl. C12N 15/82

(52) U.S. Cl. CPC C12N 15/8285
USPC 800/260; 800/302; 435/418; 800/279; 536/23.6

(57) ABSTRACT

The present invention relates to methods for breeding cotton plants containing a recombination event such that quantitative trait loci (“QTL”) on chromosome 11 are present, associated with resistance to both root-knot nematode (“RKN”) and reniform nematode (“REN”) infection (“stacked nematode resistance”). The invention further provides germplasm and the use of germplasm containing such stacked nematode resistance as a source of nematode resistance alleles for introgression into elite germplasm in a breeding program, thus producing novel elite germplasm comprising the stacked nematode resistance trait.

### CROSS-REFERENCE TO RELATED APPLICATIONS

This application claims the priority of U.S. Provisional Appl. Ser. No. 61/709,049, filed Oct. 2, 2012, the entire disclosure of which is incorporated herein by reference.

### INCORPORATION OF SEQUENCE LISTING

The sequence listing that is contained in the file named “MONS:297US.txt”, which is 55,437 bytes (measured in MS-WINDOWS), created on Sep. 13, 2013, is filed herewith by electronic submission and is incorporated herein by reference.

### FIELD OF THE INVENTION

The invention relates to methods and compositions for identifying and producing cotton plants (Gossypium sp.) with resistance to Root knot nematode, a disease associated with Meloidogyne incognita, as well as being resistant to Reniform nematode disease, caused by Rotylenchulus reniformis.

### BACKGROUND OF THE INVENTION

Plants are subject to multiple potential disease causing agents, including plant-parasitic nematodes or roundworms. Nematodes have a wide host range infecting many plant species including cotton (Gossypium sp.). There are numerous plant-parasitic nematode species, including Tylenchid nematodes, the largest and most economically important group of plant-parasitic nematodes, which include various root knot nematodes (e.g. Meloidogyne sp.; “RKN”), and reniform nematodes (e.g. Rotylenchulus sp. “REN”), among others. Such sedentary endoparasitic nematodes, including both root-knot nematodes and reniform nematodes, induce feeding sites and establish long-term infections within roots that are often very damaging to a plant, seriously affecting the ability to take up water and nutrients from soil.

### SUMMARY OF THE INVENTION

In one aspect, the invention provides a nematode resistant cotton plant comprising: a) an introgressed locus on chromosome A11 comprising an allele that confers resistance to root-knot nematodes; and b) an introgressed locus on chromosome A11 comprising an allele that confers resistance to reniform nematodes, wherein the cotton plant exhibits resistance to root-knot nematodes and reniform nematodes. In particular embodiments, the locus comprising an allele conferring resistance to root-knot nematodes is genetically linked within 10 cM of a locus selected from the group consisting of NGHIR008355362, NG0209154, NG0210828, NG0208423, NG0208500, NG0204877, NG0210025, and NO209086 on cotton chromosome A11. In another embodiment, the locus comprising an allele conferring resistance to root-knot nematodes is localized within a chromosomal interval defined by and including the terminal markers MUSB0404 and CIR316 on cotton chromosome A11; the locus comprising an allele conferring resistance to reniform nematodes is genetically linked within 10 cM of a locus selected from the group consisting of GH300, NG0210892, NGHIR008355346, NGHIR008355350, NGHIR008355351, NGHIR008355362, and CIR196; and/or the locus comprising an allele conferring resistance to reniform nematodes is localized within a chromosomal interval defined by and including the terminal markers GH300 and CIR196 on cotton chromosome A11. The locus conferring resistance to root-knot nematodes may, in one embodiment, be derived from G. hirsutum and the locus conferring resistance to reniform nematodes may be derived from G. longicalyx.

In particular embodiments, a plant provided by the invention may be defined as a G. hirsutum cotton plant. In further embodiments, a plant of the invention may comprise at least one additional gene that confers resistance to Root-knot disease caused by Meloidogyne incognita, including RKN2. in still further embodiments, a plant of the invention comprises at least one additional gene that confers resistance to Reniform disease caused by Rotylenchulus reniformis. Plants according to the invention may, in specific embodiments, be defined as an agronomically elite plant. Also provided by the invention are plant parts of any plant according to the invention, including a cell, a seed, a root, a stem, a leaf, a flower, a boll, or pollen.

In other embodiments, populations of plants or seeds according to the invention are provided. A “population of plants”, “population of seeds”, “plant population” or “seed population” refers to a group of plants or seeds comprising alleles conferring resistance to root-knot nematodes and reniform nematodes, as described herein. In one embodiment, the population comprises the same chromosomal segment comprising the comprising alleles conferring resistance to root-knot nematodes and reniform nematodes. A population of plants or seeds can include the progeny of a single breeding cross or a plurality of breeding crosses. The population members need not be identical. In one embodiment of the invention a substantially homogenous population of plants or seeds according to the invention is provided. In further embodiment of the invention, a substantially homogenous population may be defined as a population of plants that are genetically the same as one another, save for occasional genetic variation typical of plants derived through multiple generations of inbreeding. Examples of populations include, but are not limited to, those made up of about 3, 5, 10, 25, 50, 100, 250, 500, 1000, 2500, and 5000, or more individual members.

In another aspect, the invention provides a tissue culture of regenerable cells of a plant according to the invention. In one embodiment, a plant of the invention is defined as cotton line 12D0005-RENSS, a sample of seed of said line having been deposited under ATCC Accession Number PTA-13160. Seeds, plants, plant parts and derivatives of such a line and related methods thus form part of the invention. In another embodiment, a plant is provided defined as comprising a chromosomal segment comprising an RKN1 allele of Gossypium hirsutum conferring resistance to Meloidogyne incognita and a REN allele of Gossypium longicalyx conferring resistance to Rotylenchulus reniformis that is contained in said cotton line 12D0005-RENSS; wherein a sample of seed comprising the chromosomal segment was deposited under ATCC Accession Number PTA-13160. Also provided are progeny plants of any generation of a plant described herein, as well as seed of such progeny plants and seed that produces any plant described herein.

In still yet another aspect, the invention provides a method for producing a nematode resistant cotton plant comprising: a) crossing a cotton plant comprising a chromosomal segment of cotton chromosome A11 that comprises a RKN1 allele that confers resistance to root-knot nematodes with a second cotton plant that comprises a REN allele that confers resistance to reniform nematodes; b) obtaining progeny resulting from the crossing; and c) selecting at least a first progeny plant that comprises the RKN1 and REN alleles. In one embodiment, the RKN1 allele is derived from G. hirsutum and the REN allele is derived from G. longicalyx. In the method, the step of selecting may comprise marker-assisted selection, which may or may not include detecting an allele of at least one SNP marker listed in Table 3 or in Table 4. In one embodiment the step of selecting comprises identifying in said progeny plant at least a first polymorphism associated with the presence of said RKN1 allele at a locus selected from the group consisting of NGHIR008355350, NGHIR008355362, NG0210828, and NGHIR008355360. In another embodiment, the step of selecting comprises identifying in said progeny plant at least a first polymorphism associated with the presence of said REN allele at a locus selected from the group consisting of NG0210892, NGHIR008355341, NGHIR008355346, NGHIR008355350, NGHIR008355338, NGHIR008355369, NGHIR008355351, and NGHIR008355362. The method may further comprise introgressing into said progeny plant, or a progeny plant of any generation thereof that comprises said RKN1 and REN alleles, a locus on chromosome A07 comprising a RKN2 nematode resistance allele In particular embodiments, introgressing comprises marker assisted selection for said RKN2 allele. In other embodiments, the first progeny plant is a progeny plant of cotton line 12D0005-RENSS, a sample of seed of said line having been deposited under ATCC Accession Number PTA-13160.

In still yet another aspect, the invention provides a method for producing a cotton variety displaying resistance to root-knot nematodes and reniform nematodes comprising introgressing into the variety a chromosomal segment comprising an RKN1 allele and a REN allele, wherein the RKN1 allele and the REN allele both specify nematode resistance. In one embodiment, introgressing comprises selecting at least a first progeny plant that comprises a reduction in the amount of genomic DNA located between said RKN1 and REN alleles relative to a plant of a prior generation of said progeny plant. Plants and seeds produced by such a method and any other method described herein also form a part of the invention.

In still yet another aspect, the invention provides an isolated nucleic acid segment that is selected from the group consisting of SEQ IDs NO:1-175; or: a) hybridizes under conditions of 5×SSC, 50% formamide, and 42° C. with; or b) displays at least 80% sequence identity towards a nucleic acid sequence of at least 20 contiguous nucleotides comprised within any of SEQ ID NOs:1-175, wherein the segment comprises a polymorphism mapping within 20 cM of a QTL specifying resistance to reniform or root-knot nematodes. In one embodiment, such an isolated nucleic acid segment is defined selected from the group consisting of SEQ IDs NO:1-175.

In still yet another aspect, the invention provides a recombined DNA segment an RKN1 allele and a REN allele, wherein the RKN1 allele and the REN allele both specify nematode resistance, as described herein.

### BRIEF DESCRIPTION OF THE DRAWINGS

FIG. 1 depicts exemplary haplotypes of cotton lines having undergone a recombination event on chromosome 11, and comprising marker alleles linked to the RKN1 and REN QTL's defined herein.

FIG. 2 shows the shortest segment of chromosome A11 that consistently cosegregated with tolerance to reniform infection, comprising the loci corresponding to markers NGHIR008355338, NGHIR008355350, NGHIR00355340, NGHIR008355369, and NGHIR008355351, which correspond to 160.2-160.9 cM. Regions flanking that segment were not necessary for tolerance.

### BRIEF DESCRIPTION OF THE SEQUENCES

TABLE 1
SEQ ID NOs for REN and RKN1 markers, and associated primers and probes.

| | SEQ ID | | | | | | |
|---|---|---|---|---|---|---|---|
| | NO. for | | | SEQ ID | SEQ ID | | |
| | marker | Mapped | | NO for | NO for | SEQ ID | SEQ ID |
| | genomic | Chr. Pos. | SNP | fwd | rev | NO for | NO for |
| Marker Name | sequence | (cM) | Position¹ | primer | primer | probe 1 | probe 2 |
| NG0210892 | 1 | 150.7 | 323 | 36 | 71 | 106 | 141 |
| NGHIR008355358 | 2 | 152.6 | 51 | 37 | 72 | 107 | 142 |
| NGHIR008355367 | 3 | 156.4 | 165 | 38 | 73 | 108 | 143 |
| NGHIR008355341 | 4 | 157.2 | 619 | 39 | 74 | 109 | 144 |
| NGHIR008355346 | 5 | 157.5 | 82 | 40 | 75 | 110 | 145 |
| NGHIR008355350 | 6 | 160.2 | 106 | 41 | 76 | 111 | 146 |
| NGHIR008355338 | 7 | 160.5 | 188 | 42 | 77 | 112 | 147 |
| NGHIR008355369 | 8 | 160.7 | 96 | 43 | 78 | 113 | 148 |
| NGHIR008355351 | 9 | 160.9 | 207 | 44 | 79 | 114 | 149 |
| NGHIR008355347 | 10 | 163.1 | 708 | 45 | 80 | 115 | 150 |
| NG0203802 | 11 | 163.6 | 122 | 46 | 81 | 116 | 151 |
| NGHIR008355348 | 12 | 169.6 | 225 | 47 | 82 | 117 | 152 |
| NGHIR008355362 | 13 | 170.3 | 105 | 48 | 83 | 118 | 153 |
| NG0206531 | 14 | 171.3 | 354 | 49 | 84 | 119 | 154 |
| NGHIR008355363 | 15 | 172.1 | 160 | 50 | 85 | 120 | 155 |
| NGHIR008355343 | 16 | 175.1 | 152 | 51 | 86 | 121 | 156 |
| NGHIR008355354 | 17 | 177.6 | 208 | 52 | 87 | 122 | 157 |
| NG0209154 | 18 | 178.5 | 221 | 53 | 88 | 123 | 158 |
| NG0210828 | 19 | 180.1 | 356 | 54 | 89 | 124 | 159 |
| NG0204877 | 20 | 181.1 | 409 | 55 | 90 | 125 | 160 |
| NGHIR008355342 | 21 | 182.1 | 338 | 56 | 91 | 126 | 161 |
| NGHIR008355359 | 22 | 182.2 | 139 | 57 | 92 | 127 | 162 |
| NGHIR008355357 | 23 | 182.4 | 159 | 58 | 93 | 128 | 163 |
| NGHIR008355360 | 24 | 182.5 | 318 | 59 | 94 | 129 | 164 |
| NG0203802 | 25 | 163.6 | 122 | 60 | 95 | 130 | 165 |
| NG0207423 | 26 | 163.9 | 449 | 61 | 96 | 131 | 166 |
| NG0206483 | 27 | 165.5 | 209 | 62 | 97 | 132 | 167 |
| NG0206531 | 28 | 171.3 | 354 | 63 | 98 | 133 | 168 |
| NG0209154 | 29 | 178.5 | 221 | 64 | 99 | 134 | 169 |
| NG0210828 | 30 | 180.1 | 356 | 65 | 100 | 135 | 170 |
| NG0208423 | 31 | 180.1 | 166 | 66 | 101 | 136 | 171 |
| NG0208500 | 32 | 180.1 | 219 | 67 | 102 | 137 | 172 |
| NG0204877 | 33 | 181.1 | 409 | 68 | 103 | 138 | 173 |
| NG0210025 | 34 | 181.2 | 255 | 69 | 104 | 139 | 174 |
| NG0209086 | 35 | 182.4 | 525 | 70 | 105 | 140 | 175 |

¹SNP Position refers to the position of the SNP polymorphism in the indicated SEQ ID NO.

### DETAILED DESCRIPTION OF THE INVENTION

The invention provides methods and compositions relating to cotton plants comprising introgressed chromosomal regions on cotton chromosome 11, from G. hirsutum and G. longicalyx, such that the plants surprisingly display resistance to both root-knot (“RKN”) and reniform (“REN”) nematodes. Using molecular and phenotyping tests, it was unexpectedly found that cotton lines could be developed displaying resistance to both of these nematodes, due to the occurrence and identification of a rare recombination event in a portion of chromosome 11 linked to resistance genes RKN1 and REN. This allows for identification of cotton plant lines comprising separate but tightly linked genes within quantitative trait loci (“QTL”) specifying resistance to each of these nematodes. It was previously unknown whether such a combination could be made due to the close map position of these traits, or further whether the traits could be expressed in the same plant, in view of their introgression from other Gossypium sp. The invention now allows introgression of both resistance genes which may be carried out while minimizing “linkage drag” and possible deleterious phenotypes during plant breeding.

Unlike more typical cases of introgression, wherein a gene or chromosome segment of interest is introgressed from one closely-related plant line to the other, the source of REN resistance is a species (G. longicalyx, 2×, 2[F1] genome, native to Africa,) that is only distantly related to the cultivated upland cotton (G. hirsutum, 4×, 2[(AD)1] genome, native to Mexico). Furthermore, sequence divergence between plants from these distinct species in the region comprising the RKN1 and REN resistance loci of chromosome 11 was found to result in an apparent lack of synteny and a corresponding suppression of recombination in this region. Thus, an unexpected and fortuitous recombination event was necessary to allow for incorporation, in a progeny plant, of the chromosome segment containing the REN resistance locus from the F genome of G. longicalyx with the RKN1 locus in the A sub-genome of G. hirsutum.

The invention thus provides methods and compositions for identifying cotton plants (Gossypium sp. including G. hirsutum and G. barbadense) having genetic resistance to Root-knot nematode and Reniform diseases caused by Meloidogyne incognita and Rotylenchulus reniformis, respectively. Such cotton plants can be referred to as “stacked nematode-resistant” cotton plants. Methods of breeding such stacked nematode resistant cotton lines are further provided. Also disclosed herein are molecular markers that are linked to the QTL(s) contributing to resistance to RKN and REN. Through use of the markers, one of skill in the art may use marker-assisted selection to increase the degree of nematode resistance in cotton, or select plants for an increased predisposition for nematode resistance. The introgressed genomic region comprising the stacked nematode resistance genes may also be utilized in conjunction with other disease resistance genes found in cotton, to further augment the level of disease resistance and produce novel elite cotton germplasm displaying, for instance, improved resistance to RKN and REN.

Using the techniques described herein, the inventors identified a recombination event wherein the RKN1 resistance gene from a G. hirsutum source (WO 2010/025172) and the REN resistance gene from a G. longicalyx source (WO 2011/0088118), both found on chromosome 11, are tightly linked. Thus a single plant containing the RKN1 and REN resistance alleles from two different species has been identified and can be produced. Such a recombined segment may be used to breed further cotton lines and cultivars comprising these nematode resistance genes. Since these separate resistance genes map closely, it was unclear that a recombination event could be obtained that would allow alleles specifying resistance at both loci to be obtained. It was further unclear whether both of these resistance genes, from distinct sources, could be successfully expressed together in a single plant while maintaining appropriate agronomic characteristics relating to plant growth and crop yield. The genomic region comprising the stacked RKN1 and REN genes further comprises reduced introgression segment size, allowing for efficient introgression of both of these resistance traits into lines of any genotype. In view of the methods and compositions described herein, alleles conferring RKN and REN nematode resistance may be combined from any source, such individual sources being known in the art even though the ability to combine such sources could not have heretofore been predicted. The stacked nematode resistance trait may also be combined with other resistance genes, such as the RKN2 gene found on chromosome 7, and other plant disease resistance genes, to further improve the level of disease resistance in cotton. It was further found that RKN2 (disclosed in WO 2010/025172) is complementary to RKN1 in that RKN2 specifies a low level of resistance to RKN on its own, but when combined with RKN1 resistance is unexpectedly higher.

In certain embodiments, the methods are performed on progeny cotton plants of cotton line 12D0005-RENSS, having been deposited under ATCC Accession Number PTA-13160, which comprise the introgressed chromosomal regions on chromosome A11 specifying resistance to both RKN and REN.]

Introgression of a particular DNA element or set of elements into a plant genotype is defined as the result of the process of backcross conversion. A plant genotype into which a DNA sequence has been introgressed may be referred to as a backcross converted genotype, line, or variety. Such genotype, line, or variety may be an inbred or a hybrid genotype, line, or variety. Similarly a plant genotype lacking said desired DNA sequence may be referred to as an unconverted genotype, line, or variety. During breeding, one or more genetic markers linked to enhanced nematode resistance associated with the RKN1 gene derived from a G. hirsutum source on chromosome 11, as well as the REN gene derived from a G. longicalyx source, also found on chromosome 11, and which can be produced in accordance with the invention to be tightly linked and co-inherited, may be used to assist in breeding for the purpose of producing cotton plants with increased resistance to both RKN and REN. In some embodiments, one or more additional plant disease resistance traits may also be present, such as the RKN2 gene, another genetic trait for RKN resistance which is located on chromosome 7.

A skilled worker would understand that the introgression of one or more nematode resistance trait(s) into a cotton plant may be monitored by visual clues, such as by use of a disease resistance test, and/or by monitoring and breeding for the presence of molecular markers (e.g. SNP, SSR, etc.) as described herein by marker-assisted selection. An elite cotton plant of the present invention can also exhibit a transgenic trait. The transgenic trait, in particular embodiments, may be selected from the group consisting of herbicide tolerance, increased yield, insect control, fungal disease resistance, virus resistance, nematode resistance, bacterial disease resistance, mycoplasma disease resistance, modified oils production, high oil production, high protein production, germination and/or seedling growth control, enhanced animal and human nutrition, low raffinose, environmental stress resistance, increased digestibility, improved processing traits, improved flavor, nitrogen fixation, hybrid seed production, and reduced allergenicity. The herbicide tolerance can be selected, for example, from the group consisting of glyphosate, dicamba, glufosinate, sulfonylurea, bromoxynil, 2, 4, Dichlorophenoxyacetic acid, and norflurazon herbicides.

Localization of genetic markers to specific genomic regions, chromosomes, or contigs further allows for use of associated sequences in breeding, to develop additional linked genetic markers, as well as to identify the mechanism for resistance at more precise genetic and biochemical levels. It will be understood to those of skill in the art that other markers or probes which also map to the chromosomal regions as identified herein could be employed to identify plants comprising the desired loci for resistance to RKN and REN. The chromosomal regions of the present invention facilitate introgression of the stacked nematode resistance into other germplasm, preferably agronomically useful cotton germplasm. Linkage blocks of various sizes could be transferred within the scope of this invention as long as the chromosomal region enhances the nematode resistance of a desirable cotton plant, line, or variety. In certain exemplary embodiments, the linkage block of chromosome A11 comprising the RKN1 and REN loci may be about 0.5, 1, 2, 3, 5, 10, 25, 40, or 50 cM in length. Thus, the linkage block (i.e. chromosome segment, also contemplated as being present on an engineered chromosome or other DNA construct) may be transferred while minimizing linkage drag. Accordingly, it is emphasized that the present invention may be practiced using any molecular markers which genetically map in similar regions, provided that the markers are sufficiently polymorphic between the parents or mapping populations.

In particular embodiments, markers may be genetically linked to the described alleles for RKN and REN resistance which are located on cotton chromosome A11. Genetic mapping information for cotton is discussed, for instance, in Blenda et al. (BMC Genomics 7:132, 2006). In certain embodiments, the markers are within about 50 cM, 45 cM, 40 cM, 30 cM, 20 cM, 10 cM, 5 cM, 3 cM, 1 cM, or less, of the REN or RKN1 alleles defined on chromosome 11. The presence of alleles conferring resistance to REN and RKN may be identified by use of well known techniques, such as by nucleic acid detection methods utilizing probes or primers comprising a sequence selected from the group consisting of SEQ ID NOs:1-175. In certain embodiments, the method comprises detecting the presence of one or more single nucleotide polymorphisms (SNP's) given in one or more of SEQ ID NOs:1-175.

In certain embodiments, the REN and RKN1 resistance QTL of chromosome 11 is defined as the interval spanning the region defined by SNP markers GH300 and CIR316, or GH300 and CIR196, or other interval(s) the borders of which are comprised within these segments of chromosome 11 as defined in Tables 3 and 4, such as the interval spanned by markers DPL0209 and NAU2152, NG0210892 and NGHIR008355362, CIR003 and CGR5428, NGHIR008355367 and NG0203802, or NGHIR008355341 and NGHIR008355351, including an interval comprised within these exemplary segments, or linked within 20 cM, 10 cM, or 5 cM of these segments of chromosome 11. These intervals may also be defined by and include any marker locus localizing within a chromosome interval flanked by and including markers MUSB0404 and CIR316 as defined in Tables 3 and 4, or other intervals whose borders fall between, and include, exemplary markers NAU2152 and CIR316, or MGHES-016 and CIR316, or any interval linked within 20 cM, 10 cM, or 5 cM of those segments of chromosome 11.

In another aspect, the present invention provides a method of producing a stacked nematode resistant cotton plant comprising: (a) crossing a cotton line having stacked nematode resistance with a second cotton line lacking stacked nematode resistance to form a segregating population; (b) screening the segregating population, or a subsequent generation, for resistance to REN and/or RKN nematodes; and (c) selecting one or more plants having said stacked nematode resistance. By “stacked nematode resistance” is meant a cotton line comprising the REN and RKN1 alleles from G. longicalyx and G. hirsutum specifying resistance to reniform and root knot nematodes, for instance as found in cotton line 12D0005-RENSS. Thus, a progeny line (i.e. of a subsequent generation) of 12D0005-RENSS is also contemplated.

In one aspect, the cotton line having stacked nematode resistance is crossed with the second cotton line for at least two generations (e.g., creating an F₂ or BC₁S₁ population) or more. In a particular embodiment, the cotton line having stacked nematode resistance is 12D0005-RENSS, or a progeny thereof. In certain embodiments, plants are identified as resistant to RKN and/or REN prior to crossing. In one aspect, plants can be selected on the basis of partial or complete resistance to RKN and/or REN. In another aspect, the segregating population is self-crossed and the subsequent population is screened for resistance. Yet another aspect of the invention provides an isolated nucleic acid sequence comprising all or a portion of any of SEQ ID NOs:1-175 as discussed herein.

Cotton plants (and parts thereof, including seed, pollen, and ovules) generated using a method of the present invention are also provided, and can be part of or generated from a breeding program. The choice of breeding method depends on the mode of plant reproduction, the heritability of the trait(s) being improved, and the type of cultivar used commercially (e.g., F₁ hybrid cultivar, pure line cultivar, etc). Selected, non-limiting approaches for breeding the plants of the present invention are set forth below. A breeding program can be enhanced using marker assisted selection of the progeny of any cross. It is further understood that any commercial and non-commercial cultivars can be utilized in a breeding program. Factors such as, for example, emergence vigor, vegetative vigor, stress tolerance, disease resistance, branching, flowering, boll size, boll quality, and/or fiber yield and fiber length will generally dictate the choice.

As used herein, a “susceptible control cotton plant” refers to a cotton plant susceptible to RKN and REN (“nematode susceptible”) including commercially available and wild relatives of cotton plants. In one embodiment, the control cotton plant is the variety DP0935B2RF; other susceptible germplasm may also be utilized. A “resistant control cotton plant” may also be utilized when evaluating nematode resistant cotton varieties. In specific embodiments a plant that is defined as exhibiting resistance to RKN or REN exhibits a statistically significant increase in nematode resistance when compared to a nematode susceptible plant, including a susceptible control cotton plant. In one embodiment, such a resistant control is a cotton plant that is not susceptible to REN and/or RKN nematodes, but is otherwise agriculturally undesirable.

As used herein, a “female parent” refers to a cotton plant that is the recipient of pollen from a male donor line, which pollen successfully pollinates an egg. A female parent can be any cotton plant that is the recipient of pollen.

As used herein, “polymorphism” means the presence of two or more variations of a nucleic acid sequence or nucleic acid feature at one or more loci in a population of one or more individuals. The variation may comprise but is not limited to one or more base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and a tag SNP. A genetic marker, a gene, a DNA-derived sequence, a haplotype, a RNA-derived sequence, a promoter, a 5′ untranslated region of a gene, a 3′ untranslated region of a gene, microRNA, siRNA, a QTL, a satellite marker, a transgene, mRNA, ds mRNA, a transcriptional profile, and a methylation pattern may also comprise polymorphisms. In addition, the presence, absence, or variation in copy number of the preceding may comprise polymorphisms.

As used herein, “linkage” is a phenomenon wherein alleles on the same chromosome tend to segregate together more often than expected by chance if their transmission was independent.

As used herein, a “marker” is an indicator for the presence of at least one phenotype (detectable characteristic), genotype, or polymorphism. Genetic markers include, but are not limited to, single nucleotide polymorphisms (SNPs), cleavable amplified polymorphic sequences (CAPS), amplified fragment length polymorphisms (AFLPs), restriction fragment length polymorphisms (RFLPs), simple sequence repeats (SSRs), insertion(s)/deletion(s) (“INDEL”(s)), inter-simple sequence repeats (ISSR), and random amplified polymorphic DNA (RAPD) sequences. A marker is preferably inherited in codominant fashion (both alleles at a locus in a diploid heterozygote are readily detectable), with no environmental variance component, i.e., heritability of 1. A “nucleic acid marker” as used herein means a nucleic acid molecule that is capable of being a marker for detecting a polymorphism, phenotype, or both associated with stacked nematode resistance. Stringent conditions for hybridization of a nucleic acid probe or primer to a marker sequence or a sequence flanking a marker sequence refers, for instance, to nucleic acid hybridization conditions of 5×SSC, 50% formamide, and 42° C. As used herein, “marker assay” means a method for detecting a polymorphism at a particular locus using a particular method, e.g. measurement of at least one phenotype (such as a visually detectable trait, including disease resistance), restriction fragment length polymorphism (RFLP), single base extension, electrophoresis, sequence alignment, allelic specific oligonucleotide hybridization (ASO), random amplified polymorphic DNA (RAPD), microarray-based technologies, PCR-based technologies, and nucleic acid sequencing technologies, etc.

As used herein, a “desirable trait” or “desirable traits” that may be introduced into nematode resistant cotton plants by breeding may, for example, be directed to the cotton plant or boll. Desirable traits, transgenic or otherwise, to be introduced into cotton may be independently selected, and may include, for example, herbicide tolerance, increased yield, insect control, fungal disease resistance, virus resistance, nematode resistance, bacterial disease resistance, mycoplasma disease resistance, modified oils production, high oil production, high protein production, germination and/or seedling growth control, enhanced animal and human nutrition, low raffinose, environmental stress resistance, increased digestibility, improved processing traits, improved flavor, nitrogen fixation, hybrid seed production, and reduced allergenicity. Desirable boll traits, e.g. as displayed by agronomically elite lines or cultivars, and that may be independently selected include, but are not limited to, average boll weight, fiber length, fiber uniformity, fiber strength, and fiber micronaire. Any combination of traits, may be combined with a nematode resistance trait. The resulting agronomically elite nematode resistant cotton plants of the present invention surprisingly display such agronomic traits in combination with stacked nematode resistance, while lacking deleterious traits.

As used herein, “genotype” is the actual nucleic acid sequence at a locus in an individual plant. As used herein, “phenotype” means the detectable characteristics (e.g. level of nematode resistance) of a cell or organism which can be influenced by genotype.

As used herein, “typing” refers to any method whereby the specific allelic form of a given cotton genomic polymorphism is determined. For example, a single nucleotide polymorphism (SNP) is typed by determining which nucleotide is present (i.e. an A, G, T, or C). Insertion/deletions (Indels) are determined by determining if the Indel is present. Indels can be typed by a variety of assays including, but not limited to, marker assays.

As used herein, the term “haplotype” means a chromosomal region within a haplotype window defined by at least one polymorphic molecular marker. The unique marker fingerprint combinations in each haplotype window define individual haplotypes for that window. Further, changes in a haplotype, brought about by recombination for example, may result in the modification of a haplotype so that it comprises only a portion of the original (parental) haplotype operably linked to the trait, for example, via physical linkage to a gene, QTL, or transgene. Any such change in a haplotype would be included in this definition of what constitutes a haplotype so long as the functional integrity of that genomic region is unchanged or improved.

As used herein, the term “haplotype window” means a chromosomal region that is established by statistical analyses known to those of skill in the art and is in linkage disequilibrium. Thus, identity by state between two inbred individuals (or two gametes) at one or more molecular marker loci located within this region is taken as evidence of identity-by-descent of the entire region. Each haplotype window includes at least one polymorphic molecular marker. Haplotype windows can be mapped along each chromosome in the genome. Haplotype windows are not fixed per se and, given the ever-increasing density of molecular markers, this invention anticipates the number and size of haplotype windows to evolve, with the number of windows increasing and their respective sizes decreasing, thus resulting in an ever-increasing degree confidence in ascertaining identity by descent based on the identity by state at the marker loci.

As used herein, “resistance allele” means the nucleic acid sequence that includes the polymorphic allele associated with resistance to a disease.

As used herein, “cotton” means Gossypium hirsutum and includes all plant varieties that can be bred with cotton, including wild cotton species such as Gossypium longicalyx. More specifically, cotton plants from the species Gossypium hirsutum and the subspecies Gossypium hirsutum L. can be genotyped using these compositions and methods. In an additional aspect, the cotton plant is from the group Gossypium arboreum L., otherwise known as tree cotton. In another aspect, the cotton plant is from the group Gossypium barbadense L., otherwise known as American pima or Egyptian cotton. In another aspect, the cotton plant is from the group Gossypium herbaceum L., otherwise known as levant cotton. Gossypium sp. or cotton plants can include hybrids, inbreds, partial inbreds, or members of defined or undefined populations.

As used herein, the term “elite line” means any line that has resulted from breeding and selection for superior agronomic performance, as is well known in the art. Non-limiting examples of elite lines that are commercially available include DP 555 BG/RR, DP 445 BG/RR, DP 444 BG/RR, DP 454 BG/RR, DP 161 B2RF, DP 141 B2RF, DP 0924 B2RF, DP 0935 B2RF, DP 121 RF, DP 174 RF (Deltapine); ST5599BR, ST5242BR, ST4554B2RF, ST4498B2RF, ST5458B2RF (Stoneville); FM9058F, FM9180B2F, FM1880B2F, FM1740B2F (FiberMax); PHY485WRF, PHY375WRF, PHY745WRF (Acala)(PhytoGen); and MCSO423B2RF, MCS0508B2RF (Cotton States).

As used herein, a “hybrid cotton plant” includes a plant resulting directly or indirectly from crosses between populations, breeds or cultivars within the genus Gossypium. “Hybrid cotton plant” as used herein also refers to plants resulting directly or indirectly from crosses between different varieties or genotypes.

Stacked nematode resistance of a cotton plant provided herein can potentially be defined as complete resistance or partial resistance. The nematode resistance of a cotton plant provided herein can be measured by any means available in the art.

In certain embodiments of the invention, nematode resistance of a cotton plant is determined by an Infection Index method. An Infection Index method may be used to assess nematode resistance (e.g. see US 2011/0088118). This method comprises quantifying the number of target organisms in a sample of matter by comparing the amount of DNA detected with a sequence specific to a target organism to the total amount of DNA detected in the sample of matter. In one embodiment of the Infection Index Method, the DNA sequence that is specific to the target organism is the ITS1 (internal transcribed spacer 1) region 5.8 S rRNA gene of the reniform nematode Rotylenchulus reniformis. In another embodiment, the pest-specific nucleic acid sequence detected is the ITS 1 region 5.8 S rRNA gene of the root knot nematode Meloidogyne incognita. Such sequences are known (e.g. Blok, et al., J. Nematology 29:16-22, 1997). In other embodiments, the pest-specific nucleic acid sequences detected are specific to other organisms. In yet other embodiments, a funnel extraction technique may be used to collect nematode eggs associated with plant roots, in order to quantify a plant's ability to resist infection by specific pests such as nematodes.

In another aspect, nematode resistance is determined by obtaining disease ratings of symptom development after one or more rounds of inoculation or infection with RKN and/or REN.

In one embodiment of the invention, a plant is assayed for nematode resistance, partial resistance or susceptibility by quantifying the number of nematode eggs associated with a plant's roots after growth to a certain size or developmental stage, in the greenhouse or in the field.

As used herein, linkage of two nucleic acid sequences, including a nucleic acid marker sequence and a nucleic acid sequence of a genetic locus imparting a desired trait such as stacked nematode resistance, may be genetic or physical or both. In one aspect of the invention, the nucleic acid marker and genetic locus conferring nematode resistance are genetically linked, and exhibit a LOD score of greater than 2.0, as judged by interval mapping for the nematode resistance trait based on maximum likelihood methods described by Lander and Botstein, 1989 (Genetics, 121:185-199), and implemented in the software package MAPMAKER (e.g. Lander et al., Genomics 1:174-181, (1987); default parameters). Alternatively, other software such as QTL Cartographer v1.17 (Basten et al., Zmap—a QTL cartographer. In: Proceedings of the 5th World Congress on Genetics Applied to Livestock Production: Computing Strategies and Software, edited by C. Smith, J. S. Gavora, B. Benkel, J. Chesnais, W. Fairfull, J. P. Gibson, B. W. Kennedy and E. B. Burnside. Volume 22, pages 65-66. Organizing Committee, 5th World Congress on Genetics Applied to Livestock Production, Guelph, Ontario, Canada, 1994; and Basten et al., QTL Cartographer, Version 1.17. Department of Statistics, North Carolina State University, Raleigh, N.C., 2004) may be used.

Mapping of QTLs is well-described (e.g. WO 90/04651; U.S. Pat. Nos. 5,492,547, 5,981,832, 6,455,758; reviewed in Flint-Garcia et al. 2003 (Ann. Rev. Plant Biol. 54:357-374, the disclosures of which are hereby incorporated by reference). The LOD score associated with a QTL essentially indicates how much more likely the data are to have arisen assuming the presence of a resistance allele rather than in its absence. The LOD threshold value for avoiding a false positive with a given confidence, say 95%, depends on the number of markers and the length of the genome. Graphs indicating LOD thresholds are set forth in Lander and Botstein (1989), and further described by Ars and Moreno-Gonzalez, Plant Breeding, Hayward, Bosemark, Romagosa (eds.) Chapman & Hall, London, pp. 314-331 (1993), and van Ooijen (Heredity 83:613-624, 1999). In other embodiments, the marker and region conferring stacked nematode resistance are genetically linked and exhibit a LOD score of greater than 3.0, or a LOD score of greater than 6.0, 9.0, 12.0, 15.0, or 18.0. In one embodiment, the marker and region contributing to stacked nematode resistance are genetically linked and exhibit a LOD score of between about 14 and about 20. When assigning the presence of a QTL, the LOD threshold score associated with a QTL analysis as described herein may be determined to be significant at the 95% confidence level, or higher, such as at the 98% or 99% confidence level. The nucleic acid marker may be genetically linked at a distance of between about 0 and about 50 centimorgans (cM) to the stacked nematode resistance locus. In other embodiments, the distance between the nucleic acid marker and the stacked nematode resistance locus of chromosome 11 is between about 0 and about 35 cM, or between about 0 and about 25 cM, or between about 0 and about 15 cM, or between about 0 and about 10 cM, or between about 0 and about 5 cM, including less than about 4, 3, 2 or 1 cM. Thus the invention provides a cotton plant comprising an introgressed chromosomal region from chromosome 11 comprising functional REN and RKN1 loci, or a progeny plant thereof, wherein the introgressed region spans 20 cM, 10 cM, 5 cM, or 1 cM of chromosome 11 and comprises both of the RKN1 and REN QTL's.

As used herein, two nucleic acid molecules are said to be capable of hybridizing to one another if the two molecules are capable of forming an anti-parallel, double-stranded nucleic acid structure. Conventional stringency conditions are described by Sambrook et al., Molecular Cloning, A Laboratory Manual, 2nd Ed., Cold Spring Harbor Press, Cold Spring Harbor, New York (1989) and by Haymes et al., Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions which promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. In some embodiments, hybridization conditions can be high, moderate or low stringency conditions. Preferred conditions include those using 50% formamide, 5.0×SSC, 1% SDS and incubation at 42° C. for 14 hours, followed by a wash using 0.2×SSC, 1% SDS and incubation at 65° C.

The specificity of hybridization can be affected by post-hybridization washes. For example, the salt concentration in the wash step can be selected from a low stringency of about 2.0×SSC at 50° C. to a moderate stringency of about 1.0×SSC at 50° C. to a high stringency of about 0.2×SSC at 50° C. In addition, the temperature in the wash step can be increased from low stringency conditions at room temperature, about 22° C., to moderate stringency conditions at about 50° C., to high stringency conditions at about 65° C. Both temperature and salt concentration may be varied, or either the temperature or the salt concentration may be held constant while the other variable is changed. In some aspects, the wash step can be performed for 5, 10, 15, 20, 25, 30, or more minutes. In another aspect, the wash step is performed for about 20 minutes. In yet another aspect, the wash step can be repeated 1, 2, 3, 4, or more times using the selected salt concentration, temperature, and time. In another aspect, the wash step is repeated twice.

A genetic marker profile of a plant may be predictive of the agronomic traits of a hybrid produced using that inbred. For example, if an inbred plant of known genetic marker profile and phenotype is crossed with a second inbred of known genetic marker profile and phenotype it is possible to predict the phenotype of the F₁ hybrid based on the combined genetic marker profiles of the parent inbreds. Methods for prediction of hybrid performance from genetic marker data are disclosed in U.S. Pat. No. 5,492,547, the disclosure of which is specifically incorporated herein by reference in its entirety. Such predictions may be made using any suitable genetic marker, for example, SSRs, INDELs, RFLPs, AFLPs, SNPs, ISSRs, or isozymes.

The genetic linkage of marker molecules to stacked nematode resistance can be established by a gene mapping model such as, without limitation, the flanking marker model, and the interval mapping, based on maximum likelihood methods described by Lander and Botstein, 1989 (Genetics, 121:185-199), and implemented in the software packages MAPMAKER (Whitehead Institute for Biomedical Research, Cambridge Mass., USA) or QTL Cartographer (North Carolina State University, Bioinformatics Research Center) or the like.

A maximum likelihood estimate (MLE) for the presence of a marker is calculated, together with an MLE assuming no trait effect, to avoid false positives. A log₁₀ of an odds ratio (LOD) is then calculated as: LOD=log₁₀ (MLE for the presence of a trait (MLE given no linked trait)).

Selection of appropriate mapping or segregation populations can be important in trait mapping. The choice of appropriate mapping population depends on the type of marker systems employed (Tanksley et al., Molecular mapping plant chromosomes. Chromosome structure and function: Impact of new concepts J. P. Gustafson and R. Appels (eds.), Plenum Press, New York, pp. 157-173 (1988)). Consideration must be given to the source of parents (adapted vs. exotic) used in the mapping population. Chromosome pairing and recombination rates can be severely disturbed (suppressed) in wide crosses (adapted×exotic) and generally yield greatly reduced linkage distances. Wide crosses will usually provide segregating populations with a relatively large array of polymorphisms when compared to progeny in a narrow cross (adapted×adapted).

Advanced breeding lines are collected from breeding programs. These are tested for their phenotype (e.g. their disease score reactions), and genotyped for markers in the stacked nematode resistance QTL region on chromosome 11. From these data, the smallest genetic interval is identified within each QTL containing the donor parent (DP) favorable allele among the stacked nematode resistant lines.

As used herein, progeny include not only, without limitation, the products of any cross (be it a backcross or otherwise) between two plants, but all progeny whose pedigree traces back to the original cross. Specifically, without limitation, such progeny include plants that have 50%, 25%, 12.5% or less nuclear DNA derived from one of the two originally crossed plants. As used herein, a second plant is derived from a first plant if the second plant's pedigree includes the first plant.

The present invention provides a genetic complement of the cotton lines described herein. Means for determining such a genetic complement are well-known in the art.

As used herein, the phrase “genetic complement” means an aggregate of nucleotide sequences, the expression of which defines the phenotype of a plant, such as a G. hirsutum cotton plant or a cell or tissue of that plant. By way of example, a cotton plant is genotyped to determine a representative sample of the inherited markers it possesses. Markers are preferably inherited in codominant fashion so that the presence of both alleles at a diploid locus is readily detectable, and they are free of environmental variation, i.e., their heritability is close to, or equal to, 1. This genotyping is preferably performed on at least one generation of the descendant plant for which the numerical value of the trait or traits of interest are also determined. The array of single locus genotypes is expressed as a profile of marker alleles, two at each locus for a diploid plant. The marker allelic composition of each locus can be either homozygous or heterozygous. Homozygosity is a condition where both alleles at a locus are characterized by the same conditions of the genome at a locus (e.g., the same nucleotide sequence). Heterozygosity refers to different conditions of the genome at a locus. Potentially any type of genetic marker could be used, for example, simple sequence repeats (SSRs), insertion/deletion polymorphism (INDEL), restriction fragment length polymorphisms (RFLPs), amplified fragment length polymorphisms (AFLPs), single nucleotide polymorphisms (SNPs), and isozymes.

Considerable genetic information can be obtained from a completely classified F₂ population using a codominant marker system (Mather, Measurement of Linkage in Heredity: Methuen and Co., (1938)). An F₂ population is the first generation of self or sib pollination after the hybrid seed is produced. Usually a single F₁ plant is self or sib pollinated to generate a population segregating for the nuclear-encoded genes in a Mendelian (1:2:1) fashion.

In contrast to the use of codominant markers, using dominant markers often requires progeny tests (e.g., F₃ or back cross self families) to identify heterozygous individuals. The information gathered can be equivalent to that obtained in a completely classified F₂ population. This procedure is, however, often prohibitive because of the cost and time involved in progeny testing. Progeny testing of F₂ individuals is often used in map construction where error is associated with single plant phenotyping, or when sampling the plants for genotyping affects the ability to perform accurate phenotyping, or where trait expression is controlled by a QTL. Segregation data from progeny test populations (e.g., F₃ or backcrossed or selfed families) can be used in trait mapping. Marker-assisted selection can then be applied to subsequent progeny based on marker-trait map associations (F₂, F₃), where linkage has not been completely disassociated by recombination events (i.e., maximum disequilibrium).

Recombinant inbred lines (RILs) (genetically related lines; usually >F₅) can be used as a mapping population. RILs can be developed by selfing F2 plants, then selfing the resultant F3 plants, and repeating this generational selfing process, thereby increasing homozygosity. Information obtained from dominant markers can be maximized by using RILs because all loci are homozygous or nearly so. Under conditions of tight linkage (i.e., about <10% recombination), dominant and co-dominant markers evaluated in RIL populations provide more information per individual than either marker type in backcross populations (e.g. Reiter et al., 1992; Proc. Natl. Acad. Sci. (U.S.A.) 89:1477-1481). However, as the distance between markers becomes larger (i.e., loci become more independent), the information in RIL populations decreases dramatically when compared to codominant markers.

Backcross populations can be utilized as mapping populations. A backcross population (BC) can be created by crossing an F₁ to one of its parents. Typically, backcross populations are created to recover the desirable traits (which may include most of the genes) from one of the recurrent parental (the parent that is employed in the backcrosses) while adding one or a few traits from the second parental, which is often referred to as the donor. A series of backcrosses to the recurrent parent can be made to recover most of the recurrent parent's desirable traits. Thus a population is created consisting of individuals nearly like the recurrent parent, wherein each individual carries varying amounts or a mosaic of genomic regions from the donor parent. Backcross populations can be useful for mapping dominant markers particularly if all loci in the recurrent parent are homozygous and the donor and recurrent parent have contrasting polymorphic marker alleles (Reiter et al., 1992; Proc. Natl. Acad. Sci. (U.S.A.) 89:1477-1481).

Information obtained from backcross populations using either codominant or dominant markers is less than that obtained from completely classified F₂ populations because recombination events involving one, rather than two, gametes are sampled per plant. Backcross populations, however, are more informative (at low marker saturation) when compared to RILs as the distance between linked loci increases in RIL populations (i.e., about 15% recombination). Increased recombination can be beneficial for resolution of tight linkages, but may be undesirable in the construction of maps with low marker saturation.

Near-isogenic lines (NIL) created by many backcrosses to produce an array of individuals that are nearly identical in genetic composition except for the trait or genomic region under interrogation can be used as a mapping population. In mapping with NILs, only a portion of the loci polymorphic between the parentals are expected to segregate in the highly homozygous NIL population. Those loci that are polymorphic in a NIL population, however, are likely to be linked to the trait of interest.

Bulk segregant analysis (BSA) is a method developed for the rapid identification of linkage between markers and traits of interest (Michelmore, et al., 1991; Proc. Natl. Acad. Sci. (U.S.A.) 88:9828-9832). In BSA, two bulk DNA samples are drawn from a segregating population originating from a single cross. These bulk samples contain individuals that are identical for a particular trait (e.g., resistant or susceptible to a particular pathogen) or genomic region but arbitrary at unlinked regions (i.e., heterozygous). Regions unlinked to the target trait will not differ between the bulked samples of many individuals in BSA.

For highly heritable traits, a choice of superior individual plants evaluated at a single location will be effective, whereas for traits with low heritability, selection should be based on statistical analyses (e.g., mean values) obtained from replicated evaluations of families of related plants. Popular selection methods commonly include pedigree selection, modified pedigree selection, mass selection, and recurrent selection. In a preferred embodiment a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Breeding lines can be tested and compared to appropriate standards in environments representative of the commercial target area(s) for two or more generations. The best lines are candidates as parents for new commercial cultivars; those still deficient in traits may be used as parents for hybrids, or to produce new populations for further selection.

One method of identifying a superior plant is to observe its performance relative to other experimental plants and to a widely grown standard cultivar. If a single observation is inconclusive, replicated observations can provide a better estimate of its genetic worth. A breeder can select and cross two or more parental lines, followed by repeated self or sib pollinating and selection, producing many new genetic combinations.

The development of new cotton lines requires the development and selection of cotton varieties, the crossing of these varieties and selection of superior hybrid crosses. The hybrid seed can be produced by manual crosses between selected male-fertile parents or by using male sterility systems. Hybrids can be selected for certain single gene traits such as flower color, seed yield or herbicide resistance that indicate that the seed is truly a hybrid. Additional data on parental lines, as well as the phenotype of the hybrid, influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes into parent lines. These lines are used to produce new cultivars. New cultivars can be evaluated to determine which have commercial potential.

Pedigree breeding is used commonly for the improvement of self-pollinating crops. Two parents who possess favorable, complementary traits are crossed to produce an F₁. An F₂ population is produced by selfing one or several F₁'s. Selection of the best individuals in the best families is performed. Replicated testing of families can begin in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Backcross breeding and cross breeding have been used to transfer genes for a simply inherited, highly heritable trait into a desirable homozygous cultivar or inbred line, which is the recurrent parent. The source of the trait to be transferred is called the donor parent. The resulting plant obtained from a successful backcrossing program is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. After multiple backcrossing generations with selection, the resulting line is expected to have the attributes of the recurrent parent (e.g., cultivar) and the desirable trait transferred from the donor parent.

Cross breeding or backcross breeding of a stacked nematode resistant cotton plant may be conducted where the other parent (second cotton plant) is RKN and REN resistant or the other parent is not resistant to these nematodes.

Descriptions of other breeding methods that are commonly used for different traits and crops can be found in one of several available reference books (e.g., Fehr, Principles of Cultivar Development Vol. 1, pp. 2-3 (1987)).

In one aspect of the present invention, the source of RKN nematode resistance trait for use in a breeding program is derived from a G. hirsutum plant or resistant progeny thereof, as described in US Patent Application Publication 2011/0088118 or PCT Publication WO 2010/025172. In another aspect, the source of the REN resistance trait for use in a breeding program is derived from a G. longicalyx source, and REN resistant progeny thereof.

In one embodiment, the invention provides a nematode resistant cotton plant, or the seeds or other plant parts thereof, wherein the cotton plant demonstrates a reduction in symptoms relating to nematode infestation (e.g. root egg counts or plant stunting) relative to a non-resistant control plant upon inoculation or infection with REN and/or RKN. In other embodiments, a stacked nematode resistant cotton plant may also demonstrates resistance to one or more other cotton plant diseases, such as those caused by fungi, bacteria, phytoplasma, or viruses, and/or other desirable agronomic trait(s).

One aspect of the invention provides a stacked nematode resistant cotton plant, or the seeds thereof, wherein the cotton plant, expresses one, or two, or three, or more independently selected desirable traits in addition to stacked nematode resistance. In one embodiment, the “desirable trait” or “desirable traits” (transgenic or otherwise) are selected from the group consisting of: herbicide tolerance, increased yield, insect resistance, fungal disease resistance, virus resistance, nematode resistance, bacterial disease resistance, mycoplasma disease resistance, modified oils production, high seed oil production, high seed protein production, enhanced germination and/or seedling growth, enhanced animal and human nutrition, low raffinose, environmental stress resistance, drought tolerance, increased digestibility, improved processing traits, hybrid seed production, and reduced allergenicity. The herbicide tolerance can be selected from the group consisting of glyphosate, dicamba, glufosinate, sulfonylurea, bromoxynil, 2, 4, Dichlorophenoxyacetic acid, and norflurazon herbicide tolerance. In still another embodiment the “desirable trait” or “desirable traits” are selected from the group consisting of: plant height, fiber yield, boll size, boll shape, boll or fiber color, boll quality, and fiber length.

In other aspects of the invention, the plants bearing one or more desirable traits in addition to stacked nematode resistance display a greater than 10%, or a greater than 30%, or a greater than 60%, or a greater than 80% reduction in symptoms relative to a non-resistant control plant upon inoculation or infection with REN and/or RKN. The reduction in symptoms may be quantified, for instance, by comparing plant height, above ground biomass, root biomass, and/or yield versus a “susceptible” or other control line. Another aspect of the present invention is directed to a method of producing a stacked nematode resistant cotton plant comprising: crossing a cotton line having stacked nematode resistance with a second plant lacking stacked nematode resistance but capable of donating one or more of the aforementioned desirable traits.

Deposit Information

A deposit of cotton line 12D0005-RENSS, disclosed above and recited in the claims, has been made with the American Type Culture Collection (ATCC), 10801 University Blvd., Manassas, Va. 20110-2209. The date of the deposit was Aug. 15, 2012. The accession number for those deposited seeds of cotton line 12D0005-RENSS is ATCC Accession Number PTA-13160. Upon issuance of a patent, all restrictions upon the deposits will be removed, and the deposits are intended to meet all of the requirements of 37 C.F.R. §1.801-1.809. The deposits will be maintained in the depository for a period of 30 years, or 5 years after the last request, or for the effective life of the patent, whichever is longer, and will be replaced if necessary during that period.

As various modifications could be made in the compositions and methods herein described and illustrated without departing from the scope of the invention, it is intended that all matter contained in the foregoing description or shown in the accompanying drawings shall be interpreted as illustrative rather than limiting. The breadth and scope of the present invention should not be limited by any of the above-described exemplary embodiments, but should be defined only in accordance with the following claims appended hereto and their equivalents.

### EXAMPLES

### Example 1

### Identification of Recombinants that have a Reniform Nematode Resistance QTL (REN) and Root-Knot Resistance QTL (RKN1), Possessing Introgression Segments from the Resistance Donor Parents

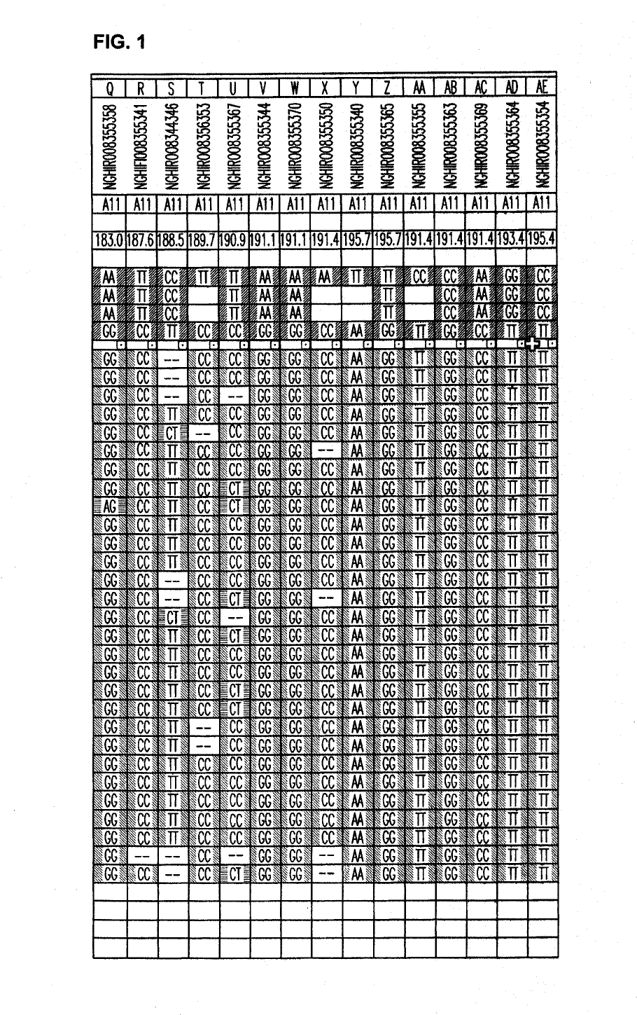

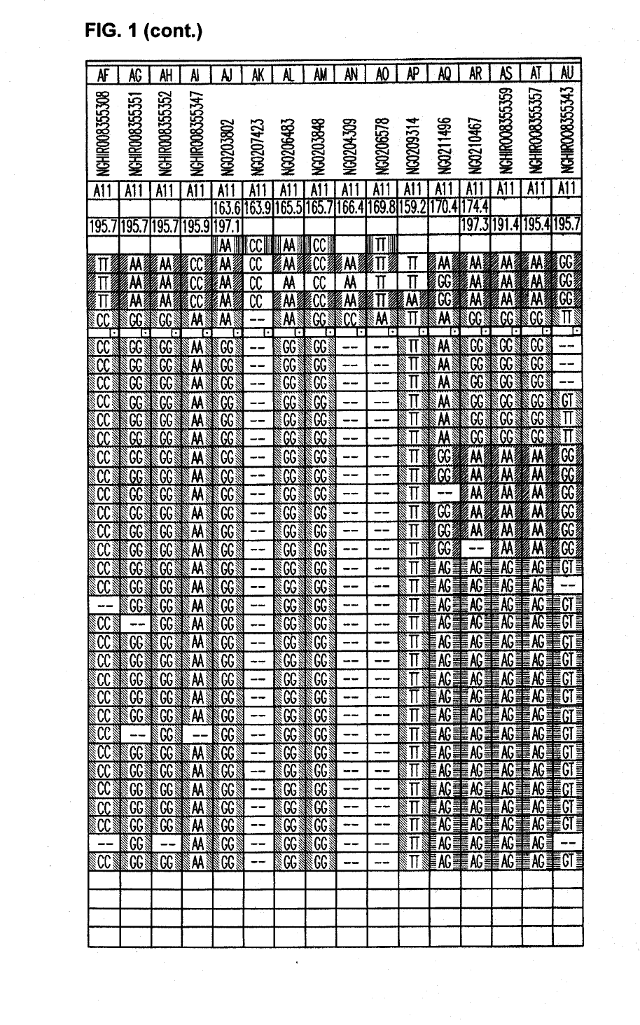

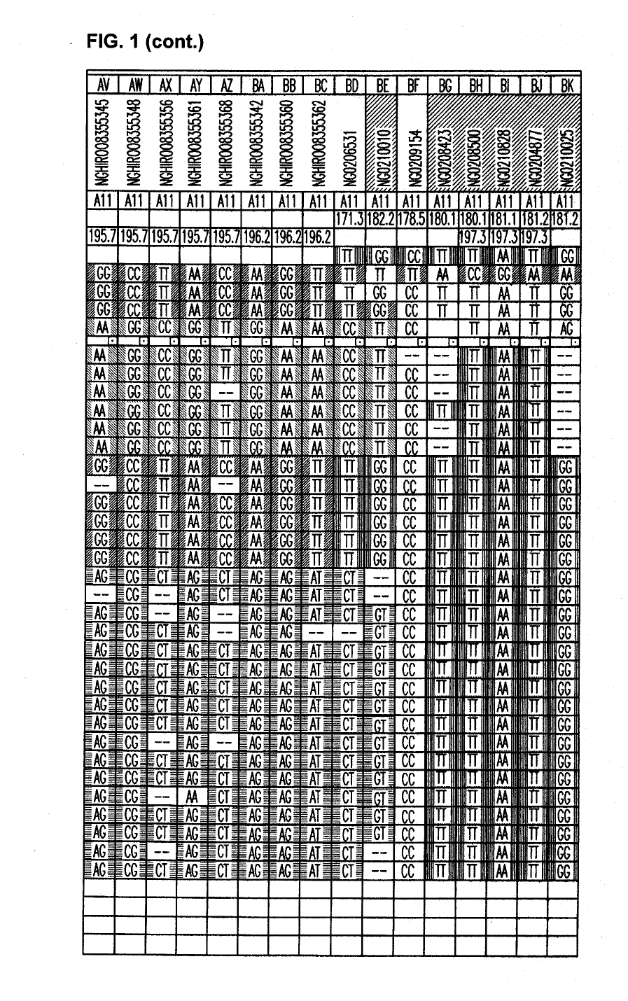

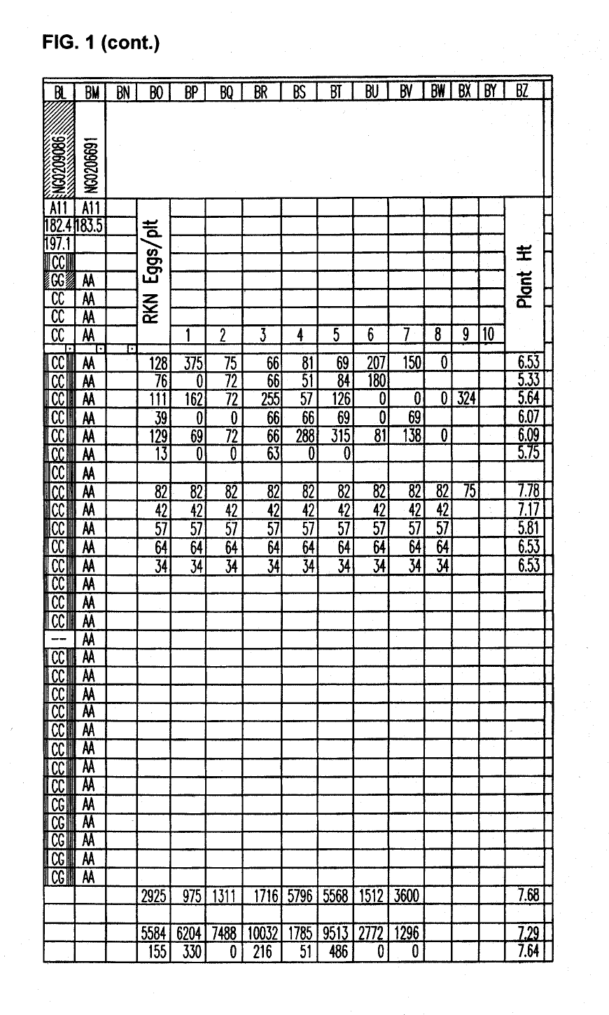

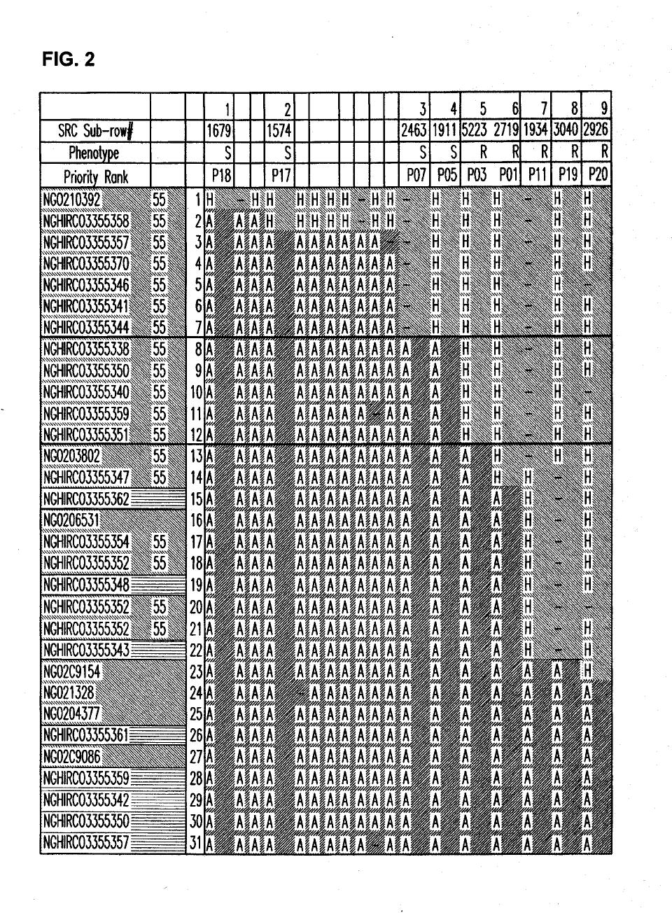

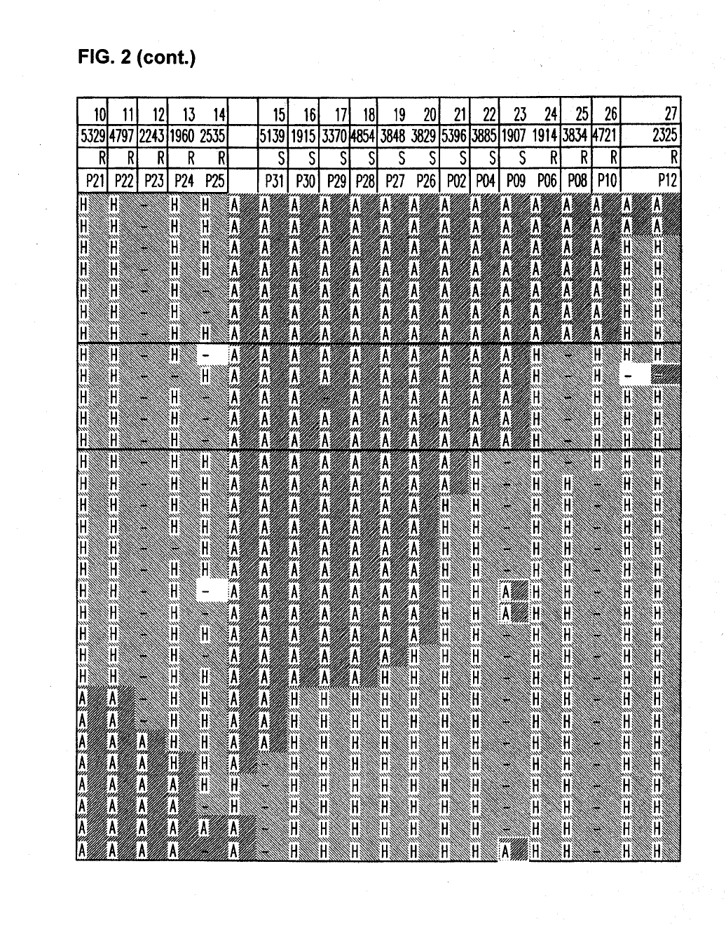

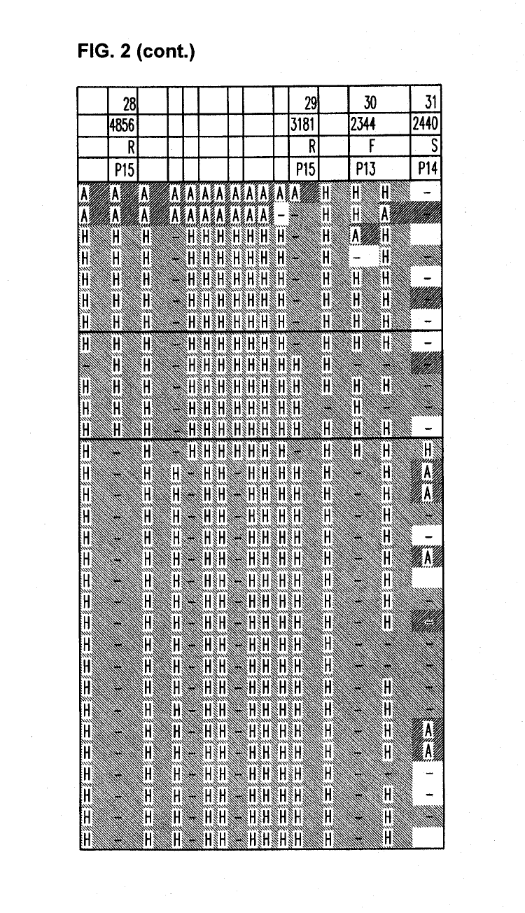

Reniform-resistant cotton line LONREN-2 was previously developed by a complex series of crosses between Gossypium hirsutum, G. longicalyx, and other germplasm (Stan et al., J. Nematol. 39:283-294, 2007; Sikkens et al., Nematropica 41:68-74, 2011), wherein the G. longicalyx parent provided a reniform nematode resistance trait mapping to cotton chromosome A11. F4 Progeny from a cross between cotton line LONREN-2, and line M240 (e.g. US 20110173713) displaying the RKN1 root knot nematode resistance trait, were screened with additional molecular markers mapping to the chromosome A11 region of RKN1 and REN. The progeny were also subjected to field evaluations for plant height. Haplotypes of selected lines are given in FIG. 1. A rare recombinant line comprising both REN and RKN1 resistance alleles was identified and studied in further progeny lines. The lines with haplotypes defining the shortest segments containing both of the resistance genes (i.e. for resistance to each of reniform and root knot nematodes) were also screened for resistance to REN and RKN. Table 2 and FIG. 1 demonstrate that the mean number of RKN eggs isolated from roots of resistant plants was significantly reduced. Resistant progeny plant heights for some lines were not significantly different from control parental lines, demonstrating no stunting.

TABLE 2
Phenotypic data from representative lines comprising recombination
event on chromosome 11 for, compared to controls.

| | | | | Plant height |
|---|---|---|---|---|
| | # of | # of eggs per | # of eggs per | (mean, in |
| Line | replicates | plant (range) | plant (mean) | inches) |
| 1 | 8 | 0-375 | 128 | 6.53 |
| 2 | 6 | 0-180 | 76 | 5.33 |
| 3 | 9 | 0-324 | 111 | 5.64 |
| 4 | 7 | 0-69 | 39 | 6.07 |
| 5 | 8 | 0-315 | 129 | 6.09 |
| 6 | 5 | 0-63 | 13 | 5.75 |
| 7 | 9 | 0-360 | 82 | 7.78 |
| 8 | 8 | 0-138 | 42 | 7.17 |
| 9 | 8 | 0-132 | 57 | 5.81 |
| 10 | 8 | 0-162 | 64 | 6.53 |
| 11 | 8 | 0-132 | 34 | 6.53 |
| Resistant control | 7 | 975-5796 | 2925 | 7.68 |
| LONREN-2 | | | | |
| Susceptible | 7 | 1296-10032 | 5584 | 7.29 |
| control DP0935 | | | | |
| Resistant control | 7 | 0-486 | 155 | 7.64 |
| M315 | | | | |

### Example 2

### Construction of Additional Mapping Populations, and QTL Analysis of REN

In order to avoid or minimize potential linkage drag relating to presence of the introgressed nematode resistance genes, and to more accurately map the length and location of the chromosomal region introgressed from the rare recombinant, an additional mapping population was prepared. The BC1F1 generation of two crosses involving DP1048B2RF and a sib of DP1048B2RF with the LONREN-2 resistance allele was evaluated, and 268 possible recombinants were identified from genotype screening of 3809 plants. These were re-genotyped and 31 true recombinants were selected. 35 BC1F2 seed from each recombinants were planted, genotyped and phenotyped on an individual plant basis, and 15 recombinant lines were selected as having resistance. Marker sequence was determined based on the linkage analysis and co-segregating markers were arranged under the assumption of the occurrence of only a single cross over event. Markers associated with the resistance genotype that were common among 15 recombinant lines indicated the region of interest.

The segregating population consisted of 899 BC1F2 plants from an original cross between the recurrent parent DP1048B2RF and LONREN-2. This population was genotyped with 27 SNP markers and JoinMap ver. 4 (Kyazma, B. V., Wageningen, N L) was used to determine the order of the markers in the chromosomal region of interest based on segregation data of the mapping population. The linkage map was constructed using regression mapping and Kosambi's mapping function. The grouping of markers was performed at a logarithm of odds (LOD) threshold≧3. The order of six markers previously mapped was used as fixed-order prior to mapping. The positions of the BSA-generated SNP on the consensus map markers were then linearly interpolated based on their relative positions on the de novo map compared to the six common markers.

For QTL analysis, extreme outliers were identified and removed prior to data analysis. A logarithm transformation was applied to approximate normality, and QTL mapping was performed using the R/qt1 package (Broman et al., Bioinformatics 19:889-890, 2003). Both the composite interval mapping and the multiple QTL mapping methods were used to estimate the QTL location. A genome-wide significance threshold was estimated after 1000 permutations. QTL confidence interval was estimated using the 1.5-LOD support interval and the Bayesian credible interval estimate method. Other QTL parameters including the percent of variation explained, and the QTL effect were estimated. The REN resistance gene mapped to a position at about 159.7 cM on cotton chromosome 11 in this analysis, as shown in Table 3.

TABLE 3
Exemplary genetic markers comprising SNP linked to REN resistance alleles.

| | SEQ ID | Chr. Pos. | SNP | Allele | Allele | | |
|---|---|---|---|---|---|---|---|
| Marker Name | NO. | (cM) | Position¹ | 1 | 2 | p-val | Effect* |
| BNL836 | | 134.7 | | | | | |
| GH300 | | 138.4 | | | | | |
| NG0210892 | 1 | 150.7 | 323 | C | T | <2.20E−16 | −776.5 |
| DPL0209 | | 151.8 | | | | | |
| NGHIR008355358 | 2 | 152.6 | 51 | G | A | <2.20E−16 | −828.5 |
| CIR003 | | 156.0 | | | | | |
| NGHIR008355367 | 3 | 156.4 | 165 | C | T | <2.20E−16 | −1112.1 |
| NGHIR008355341 | 4 | 157.2 | 619 | C | T | <2.20E−16 | −1115.5 |
| NGHIR008355346 | 5 | 157.5 | 82 | C | T | <2.20E−16 | −1142.1 |
| REN | | 159.7 | | | | | |
| NGHIR008355350 | 6 | 160.2 | 106 | T | C | <2.20E−16 | −1352.2 |
| NGHIR008355338 | 7 | 160.5 | 188 | T | C | <2.20E−16 | −1362.3 |
| NGHIR008355369 | 8 | 160.7 | 96 | C | A | <2.20E−16 | −1362.4 |
| NGHIR008355351 | 9 | 160.9 | 207 | A | G | <2.20E−16 | −1358.8 |
| MUSB0404 | | 162.4 | | | | | |
| NGHIR008355347 | 10 | 163.1 | 708 | C | A | <2.20E−16 | −1112.4 |
| NG0203802 | 11 | 163.6 | 122 | G | A | <2.20E−16 | −1123.5 |
| CGR5428 | | 167.1 | | | | | |
| NGHIR008355348 | 12 | 169.6 | 225 | G | C | <2.20E−16 | −1083.7 |
| NAU2152 | | 170.0 | | | | | |
| NGHIR008355362 | 13 | 170.3 | 105 | T | A | <2.20E−16 | −1079.7 |
| NG0206531 | 14 | 171.3 | 354 | C | T | <2.20E−16 | −1077.4 |
| NGHIR008355363 | 15 | 172.1 | 160 | C | G | <2.20E−16 | −1075.2 |
| CGR6830 | | 173.5 | | | | | |
| NGHIR008355343 | 16 | 175.1 | 152 | T | G | <2.20E−16 | −934.7 |
| NGHIR008355354 | 17 | 177.6 | 208 | T | C | <2.20E−16 | −672.9 |
| NG0209154 | 18 | 178.5 | 221 | C | T | 5.422E−15 | −555.3 |
| MGHES-016 | | 178.5 | | | | | |
| NAU2016 | | 178.5 | | | | | |
| MUCS088 | | 179.4 | | | | | |
| NG0210828 | 19 | 180.1 | 356 | G | A | 0.00002874 | −254.7 |
| CIR196 | | 180.6 | | | | | |
| NG0204877 | 20 | 181.1 | 409 | A | T | 0.00003816 | −264.0 |
| NGHIR008355342 | 21 | 182.1 | 338 | G | A | 0.0002072 | −65.9 |
| NGHIR008355359 | 22 | 182.2 | 139 | G | A | 0.0001226 | −73.2 |
| NGHIR008355357 | 23 | 182.4 | 159 | A | G | 0.0004274 | −82.1 |
| NGHIR008355360 | 24 | 182.5 | 318 | A | G | 0.0007 | −73.5 |
| CIR316 | | 202.7 | | | | | |

¹SNP Position refers to the position of the SNP polymorphism in the indicated SEQ ID NO.; listed markers without associated SEQ ID NOs represent markers available, for instance, at the Cotton Marker Database (www.cottonmarker.org; Blenda et al., BMC Genomics 7: 132, 2006). “REN” refers to the assigned map position of this resistance gene.
*number of juvenile reniform per 3 g soil sample

A similar analysis was performed to map the location of the RKN1 resistance gene, which was found to map at about position 181.8 on cotton chromosome 11.

TABLE 4
Exemplary genetic markers comprising SNP linked to a RKN1 resistance allele.

| | SEQ ID | Chr. Pos. | SNP | Allele | Allele | | |
|---|---|---|---|---|---|---|---|
| Marker Name | NO. | (cM) | Position¹ | 1 | 2 | p-val | Effect* |
| MUSB0404 | | 162.4 | | | | | |
| NG0203802 | 25 | 163.6 | 122 | A | G | 0.2442 | 69.5 |
| NG0207423 | 26 | 163.9 | 449 | C | T | 0.2562 | 66.9 |
| NG0206483 | 27 | 165.5 | 209 | A | G | 0.4278 | −0.6 |
| CGR5428 | | 167.1 | | | | | |
| NAU2152 | | 170.0 | | | | | |
| NG0206531 | 28 | 171.3 | 354 | C | T | 0.04655 | 60.7 |
| CGR6830 | | 173.5 | | | | | |
| NG0209154 | 29 | 178.5 | 221 | C | T | 2.12E−05 | −275.3 |
| MGHES-016 | | 178.5 | | | | | |
| NAU2016 | | 178.5 | | | | | |
| MUCS088 | | 179.4 | | | | | |
| NG0210828 | 30 | 180.1 | 356 | A | G | 5.72E−06 | −293.6 |
| NG0208423 | 31 | 180.1 | 166 | A | T | 1.23E−05 | −293.6 |
| NG0208500 | 32 | 180.1 | 219 | C | T | 1.07E−06 | −293.6 |
| CIR196 | | 180.6 | | | | | |
| NG0204877 | 33 | 181.1 | 409 | A | T | 2.60E−10 | −369.2 |
| NG0210025 | 34 | 181.2 | 255 | A | G | 1.17E−15 | −370.3 |
| RKN1 | | 181.8 | | | | | |
| NG0209086 | 35 | 182.4 | 525 | C | G | 2.20E−16 | 408.5 |
| CIR316 | | 202.7 | | | | | |

¹SNP Position refers to the position of the SNP polymorphism in the indicated SEQ ID NO.; listed markers without associated SEQ ID NOs represent markers available, for instance, at the Cotton Marker Database (www.cottonmarker.org; Blenda et al., BMC Genomics 7: 132, 2006). “RKN1” refers to assigned map position of this resistance gene.
*number of eggs per g of root

### Example 3

### Linkage Mapping of the RKN1-REN Recombinant

A bi-parental mapping population was developed by crossing a RKN susceptible-line and the RKN-resistant line DP174RF. Bulk harvested F1 seed was planted and F2 and F3 seed was harvested from greenhouse grown plants using the single seed descent method (SSD). Individual F3 plants were harvested in the greenhouse to give F4 seed. F4 seed was planted as progeny rows and individual plants within a progeny row were selected. Seed from homozygous plants within a progeny row were bulked to give F5 seed, and F6 and F7 seed was grown. A total of 128 F7 lines were developed for this mapping population.

A previous RKN mapping study indicated two major QTL's associated with RKN genes on linkage groups A11 and A07. Using the known RKN-1/2 associated haplotypes from this study, 22 F3-7 lines from the DP174RF-derived mapping population were selected for phenotyping to represent unique RKN-1/2 haplotype combinations. Phenotyping tests also included resistant and susceptible parents, and checks. Ten replications per entry were planted in a growth chamber and phenotyped. Data from these 22 F3-7 lines was used for QTL mapping.

QTL analysis was performed on a F4 population comprising 217 individuals originating from a cross between the RKN susceptible parent and the RKN resistant parent DP174RF. The mapping population was genotyped with 24 informative markers distributed across chromosomes A07, A11 and D02. The phenotypic data was checked for extreme outliers and normality. The multiple QTL mapping (MQM) method available in the R/qt1 package was used to identify chromosome regions associated with the variation of the root knot nematode egg count. A genome-wide significance threshold was estimated after 1000 permutations. QTL confidence interval was estimated using the 1.5-LOD support interval and the Bayesian credible interval estimate method. QTL parameters including the percent of variation explained and the QTL effect were estimated.

1. A nematode resistant cotton plant comprising:
a) an introgressed locus on chromosome A11 comprising an allele that confers resistance to root-knot nematodes; and
an introgressed locus on chromosome A11 comprising an allele that confers resistance to reniform nematodes,
wherein the cotton plant exhibits resistance to root-knot nematodes and reniform nematodes.

2. The cotton plant of claim 1, wherein the locus comprising an allele conferring resistance to root-knot nematodes is:
a) genetically linked within 10 cM of a locus selected from the group consisting of NGHIR008355362, NG0209154, NG0210828, NG0208423, NG0208500, NG0204877, NG0210025, and NO209086 on cotton chromosome A11; or
b) localized within a chromosomal interval defined by and including the terminal markers MUSB0404 and CIR316 on cotton chromosome A11.

3. (canceled)

4. The cotton plant of claim 1, wherein the locus comprising an allele conferring resistance to reniform nematodes is:
a) genetically linked within 10 cM of a locus selected from the group consisting of GH300, NG0210892, NGHIR008355346, NGHIR008355350, NGHIR008355351, NGHIR008355362, and CIR196; or
b) localized within a chromosomal interval defined by and including the terminal markers GH300 and CIR196 on cotton chromosome A11.

5. (canceled)

6. The cotton plant of claim 1, wherein the locus conferring resistance to root-knot nematodes is derived from G. hirsutum.

7. The cotton plant of claim 1, wherein the locus conferring resistance to reniform nematodes is derived from G. longicalyx.

8. The cotton plant of claim 1, defined as a G. hirsutum cotton plant.

9. The cotton plant of claim 1, further defined as comprising:
a) at least one additional gene that confers resistance to Root-knot disease caused by Meloidogyne incognita; or
b) at least one additional gene that confers resistance to Reniform disease caused by Rotylenchulus reniformis.

10. The cotton plant of claim 9, wherein the additional gene that confers resistance to Root-knot disease comprises RKN2.

11. (canceled)

12. The cotton plant of claim 1, defined as an agronomically elite plant.

13. A population of plants according to claim 1.

14. A part of the cotton plant of claim 1, wherein the part comprises at least a first cell of said cotton plant.

15. The plant part of claim 14, wherein the part is a cell, a seed, a root, a stem, a leaf, a flower, a boll, or pollen.

16. A seed that produces the plant of claim 1.

17. A population of the seed of claim 16.

18. A tissue culture of regenerable cells of cotton line 12D0005-RENSS, a sample of seed of said line having been deposited under ATCC Accession Number PTA-13160.

19. The cotton plant of claim 1, wherein the plant comprises a chromosomal segment that comprises an RKN1 allele of Gossypium hirsutum conferring resistance to Meloidogyne incognita and a REN allele of Gossypium longicalyx conferring resistance to Rotylenchulus reniformis; wherein a sample of seed comprising the chromosomal segment was deposited under ATCC Accession Number PTA-13160.

20. A progeny cotton plant of any generation of the plant of claim 19, comprising said chromosomal segment.

21-36. (canceled)

37. A seed that comprises a chromosomal segment comprising an RKN1 allele of Gossypium hirsutum conferring resistance to Meloidogyne incognita and a REN allele of Gossypium longicalyx conferring resistance to Rotylenchulus reniformis; wherein a sample of seed comprising the chromosomal segment was deposited under ATCC Accession Number PTA-13160.

38. A method for producing a nematode resistant cotton plant comprising:
a) crossing a cotton plant comprising a chromosomal segment of cotton chromosome A11 that comprises a RKN1 allele that confers resistance to root-knot nematodes with a second cotton plant that comprises a REN allele that confers resistance to reniform nematodes;
b) obtaining progeny resulting from the crossing; and
c) selecting at least a first progeny plant that comprises the RKN1 and REN alleles.

39. The method of claim 38, wherein the RKN1 allele is derived from G. hirsutum and the REN allele is derived from G. longicalyx.

40. The method of claim 38, wherein the step of selecting comprises detecting an allele of at least one SNP marker listed in Table 3 or in Table 4.

41. The method of claim 38, wherein said step of selecting comprises:
identifying in said progeny plant at least a first polymorphism associated with the presence of said RKN1 allele at a locus selected from the group consisting of NGHIR008355350, NGHIR008355362, NG0210828, and NGHIR008355360; or
identifying in said progeny plant at least a first polymorphism associated with the presence of said REN allele at a locus selected from the group consisting of NG0210892, NGHIR008355341, NGHIR008355346, NGHIR008355350, NGHIR008355338, NGHIR008355369, NGHIR008355351, and NGHIR008355362.

42. The method of claim 38, further comprising introgressing into said progeny plant, or a progeny plant of any generation thereof that comprises said RKN1 and REN alleles, a locus on chromosome A07 comprising a RKN2 nematode resistance allele.

43. The method of claim 42, wherein said introgressing comprises marker assisted selection for said RKN2 allele.

44. The method of claim 38, wherein the first progeny plant is a progeny plant of cotton line 12D0005-RENSS, a sample of seed of said line having been deposited under ATCC Accession Number PTA-13160.

45. A plant produced by the method of claim 38, or a progeny plant thereof

46. A method for producing a cotton variety displaying resistance to root-knot nematodes and reniform nematodes comprising introgressing into the variety a chromosomal segment comprising an RKN1 allele and a REN allele, wherein the RKN1 allele and the REN allele both specify nematode resistance.

47. The method of claim 46, wherein said introgressing comprises selecting at least a first progeny plant that comprises a reduction in the amount of genomic DNA located between said RKN1 and REN alleles relative to a plant of a prior generation of said progeny plant.

48. A plant produced by the method of claim 46, or a progeny plant thereof.

49. The seed of claim 37, wherein the seed is of cotton line 12D0005-RENSS, a sample of seed of said line having been deposited under ATCC Accession Number PTA-13160.

50. A plant grown from the seed of claim 49.

51. An isolated nucleic acid segment that is selected from the group consisting of SEQ IDs NO:1-175; or
a) hybridizes under conditions of 5×SSC, 50% formamide, and 42° C. with; or
b) displays at least 80% sequence identity towards a nucleic acid of at least 20 contiguous nucleotides comprised within any of SEQ ID NOs:1-175, wherein the segment comprises a polymorphism mapping within 20 cM of a QTL specifying resistance to reniform or root-knot nematodes.

52. An isolated nucleic acid segment of claim 51, further defined as being selected from the group consisting of SEQ IDs NO:1-175.


SEQUENCE LISTING (175 sequences)

SEQ ID NO: 1 - DNA, 591 bp, Gossypium hirsutum
  misc_feature (323)..(323): n is a, c, g, or t
gaatgcctcacttgcctaatttattgctttgactctttgttgtatgctatttgacttttaaccaccatcgaatataaatatataattattacaaatagtttagaagattaaataattatatttatattcaacccttttgaatattcctcggaaagttctttgaacaaagcacaaaccatgaaggttacctgtgctcgtcaagaagaaaccacgaaatttcactgtttcacaccaaagctccaccaccaagtttcccaattctaaccaattttgattaaactgagattttcccaatgcaagtgacccaattctaaatggtcttnttatgttttcaagccaagctccgagcagcaaggttggtggtccaagttcagaccctaaccaaaacagctacccgaggggctctatcgatcggaatggacacactcattcgtagacgcagaaggtctcgttgtcatcttcgctttcctattctacttctgctactttcattgatcggaagggttatcggagtcgtacttggattgttgatcaccagaatgttgatgagaaggcaataatctacataccataggttggaaattgtattag

SEQ ID NO: 2 - DNA, 574 bp, Gossypium hirsutum
  misc_feature (51)..(51): n is a, c, g, or t
atctagcatgactataactgcaccaggctatattacttggactcgggaatnagtatagtatatgggtatatatccaacaagagcatgctcaatttttctaagtttttttcacatgttggtggatacctacatccaaatatgtatcaaacatgagtgtcggacatatataccttaacaaaaatgaagagttttaagtaatgtatgcattgggtagaaatgacaggtttgtgtataaaaatactgaaaacagctggtcaagtgaataatattggtaccggggtattatcacccaaaaaagaaagcttgaaatgttcaaaatagaaataccattttatgcttctccaacattctctcttacatgtcaaataaacttcacaaagacttaaaacataagtatcgatttgttatataatgaaagcaaaatataagaacctgaaactaaaccaattctctgaagcagtgtatctgatgaccactaggtcgatcatacaattcgactgaacactaatactaataacataaaagaccagcaaagtaacagttccaaagcaaatcaaatttgtataatttattc

SEQ ID NO: 3 - DNA, 281 bp, Gossypium hirsutum
  misc_feature (165)..(165): n is a, c, g, or t
atattggctattgaatcatgtaaacctcaagaactcatgtgcatcttcctggcttccatcacccatctgacaattaatacttcggatgtgcgaaaggatcctactagaagacaagggacccccactctctcttaacaacatcacatgttgttcaagctcacacanaagacaccaatctttcccataacctacaatgaattaaaaagttatgctatgcatgtggtcaaacagtgacatcattctcatgtacaaataacaaacaagtgaacataaaaattttt

SEQ ID NO: 4 - DNA, 708 bp, Gossypium hirsutum
  misc_feature (619)..(619): n is a, c, g, or t
aagagacaggtcaaatattggtatattctatcccttagctgctgcaatgtattattgttgttgttgaatgtttttaaaatatggaattaatgctaatagcagcatttcggaataaaaatccacgactaagctagatttcatcgatgataaaatgttcttgttgttacgggttgattgttttgaacaaagcattatgggttgatttatttattattgatttgattgttgaaagttttttggatgcaataggttgttaggggttgaaccttgatcatcggaactaaagtctagcattctactgacaaaccacttgattgttgaaactttgctgaaatttcttgttgatttaatttctttcccaattcattctactttccatgagcattttattctggctttcttcggagacttgcttggtttcctgctcttcctaaacattcttttgtcatttggattaataacctgcctattattagtgctccataaacgaggcgagtggagataaggggaaaagaagggagggaagagttccagaagatgacattgaagtcttaataaaataaacatgtaataatatatgttaggaacaaccatctgttgaaatcttttgctctttgcngcccaactgtatgtcataatagattgctggattttccgcaagttctcatcaagttgatctctctctttttccatatatatctgtctctt

SEQ ID NO: 5 - DNA, 174 bp, Gossypium hirsutum
  misc_feature (26)..(26): n is a, c, g, or t
agttaccccctggcccatgaaggtangcagtatttcttaatatgacacagcattttcttacactgttttgttctggccttangaaaaataaaaagatgagatatagggtttatagcggttnagtacaatatatttgattatttatgttattctttttcccgagtagccttggtg

SEQ ID NO: 6 - DNA, 347 bp, Gossypium hirsutum
  misc_feature (72)..(72): n is a, c, g, or t
gtgtactttatatcagaagatttcaaccagtagatctccaataaacttaaatggttttgaatgttgaatgcngaagcaaaatataacatacagtgggctttgagtngacattatccataacaatttctcgtatttttccttgtccaatttcaaattcggaccgcagatctctggtaggattagtggcattatgcacagcagaaggagcctctgtaatatttgattgnaagtttaacttacaacttgggttgagaatattaacctggaancaaaacataacagatnaaaataganacaaagcgatcaacaaagttggagaaacgaggctaaaatgtctctacctcaca

SEQ ID NO: 7 - DNA, 624 bp, Gossypium hirsutum
  misc_feature (188)..(188): n is a, c, g, or t
ggccctcagcgctggtacctacgaggccaacagctgctggtagaggccggggcattaggttgatagatttagaccccgaaccttgtcaggttcttccaggggctttacctttggttgctgctgaaccggcggcttttaaccgagtagaggtggtggcagataaagatattgcaatggagggtaggagnggtgacaaaatagttggagttgatgaagaagctagtacaaccccggttcctgaaagggtatctccaaagtgtttgccatatgtttcctttttaaatatatttattttttacttaaatcttccatagtaagttgcttattcccgggacaccttacttttggcaagaaaaccggttaaatacagttttttttcgtatccttcatgacagcatttcaagaatgcttcaactaattggataatcatttctacttctattgttcatgaaagtgagggaaccagtgttttaactttcttaccaactataacgtacgtagttattttgttgtttctctcaatgcataccatttttattatttgttgcaggtacaagtgggtaattctcctgtatataaggtagaaaggaaactgggaaagggtggttttggccaggtttatgt

SEQ ID NO: 8 - DNA, 209 bp, Gossypium hirsutum
  misc_feature (96)..(96): n is a, c, g, or t
aaatacgggtattgttcgcttttctcttttcaagtttttctatgcatttagagagtccttgtaggatcatatccccataacaacgtctagataagnatcaaaacacggatacttcaagaaaactaaagaatcagagtaatatagtttccaatgaactaaaccgattcatgttgacttcaactaaggcatacttgcagttagacacatac

SEQ ID NO: 9 - DNA, 459 bp, Gossypium hirsutum
  misc_feature (72)..(72): n is a, c, g, or t
cattaacattaagttgaccttgttcatgttaacagcaatatatacgcattgtcatatagaaattcatggtgntttnttttttaatggatgaattgtagaagatgattcctcatttatcctttaccaactaattgcgttacttttgcaggttttactggcatagatgatccttacgaaccacctttgaactgtgaggtatgtgtttanttgaatacggaaattattagcggcggcattgtgaattaaaattaggctattattctgatataaatactgctagtttatgggtaatcgttttcctcctttatagatagaactaaatcagaaagatggagtttgtcccacacctagtgccatggctggggaagtaattacttacttggaggacaaaggatatctgcaaggttagcgaccaattctcggttgtcatggtcgagcattctcagtcgagttcaattg

SEQ ID NO: 10 - DNA, 958 bp, Gossypium hirsutum
  misc_feature (708)..(708): n is a, c, g, or t
taagtataaagatattgaatgatcaacatgtgtgatatattatgttaaaaaaacacacatgtatgatataccatatcactttgttccaattcagaatcaaaaggaagatcaaatgggttggttggtttaagatcagcagcttgtgtctctttctctccctgcaaaattaagtttagaacctaaattagattaaagaagagctcaataagaatccatataatataaagaacaaaagaaccagtaagttaaaagaacaaaccggaagtggattcattgatggagcatataatggtgctatgccattctctgatggtagagtagcagcaggaaaaccaatatgggagtcagcagtttggataccataattatcatggttctgcaagaatagatattttaaacataaatacaagaaatgtgaactttttggcataattagaaataaaagtagcaaacctctgaaactgctaagcccaaatgttcatcagtggtggccatatgttcgtatgctactacttgaggttcaagaggaagtgattcaaccgaatcattaaaagcattccatgactgttcaaagacaaagggtcacttgagcatgcaattgacaggagagaaaattttatatttcaaaccgtaaccatatgtagaccaagcaataaagccgttgaataaatgcatgcttaaagcatctctttcttattcttcacaaanctgcatgtgaaatggcttacctgagtgccagttgcaacagggggggcttgancctcatgtccatcttgccatttagtagacattgctgaagaatccccaaaagcactaaaattttcaaaaggtgaccactgcatggttgtgtttatggatgactgttggtcaaacttcactgacaaatcttcatcactgggcatcacagcaggtaaaagatttttggatactggatcagatgctgcaggtcctgtctctt

SEQ ID NO: 11 - DNA, 450 bp, Gossypium hirsutum
  misc_feature (122)..(122): n is a, c, g, or t
cgctgctctaattgtgtccagctcaaccacaacaatatgatttgagctgtcaccgttgtttgattgattgaaaagaccaagatactggctgggaagagctccaggaattctattattaggcnagattaaaaaggctaatccatggccagacaaagtagggtattcctccggtacaattgcaaaaaagaatgtggtcgaaaaagagaaaacactaccatttgtggagttcttgaattggattggattcttgtagaagatgtgacctgttgattggattgtagaattagtcagttttaagagcccacttgaatctacgcctgcaactccatcaacattcaagtaaccattgaaactgaactgaccctgattgatatctgaagatgcaaggttcaggaggaaaagcaacacaagcatgatcaagcaagacatcattacacacacaaaaaaaaa

SEQ ID NO: 12 - DNA, 756 bp, Gossypium hirsutum
  misc_feature (225)..(225): n is a, c, g, or t
ctctagagtcgacctgcagctctttaagtaatctgttatttcaactaacagacagtaatttgcagcagcaattatgattatgcaattgttttcagccaaagaaaaaaaggaaaagcattttaaaaatcaatataaatgatctcaagaaataacattcatatgtcacgaagctagaacattcacggcccctgacgcatgttctagccccgtgacgcatacatgttnaagcttaaaacaaagtagtaagatgatgcgatacatgaatggccaattctatataaacacnaggaagaagggacaaaatttactattttatttagggtgatggatcaaatttattttccatctttgcaagtgtgcaaattgtgcttctaattttattgaaaatgtcaaatttacaattggggtattctacagaatttttcttgccattatcattaaccttttttaaccaaattcaggaacttgggaataataatattcgaaagaggacacaataagaaggatatcatcttggatgaataaatgcaaaaagaagttaatgaaatcaaagatagcatgcctgtaactccactaaatctattgcagagcttttcaacaagtgattccatttgtttgtcctgcaaaatatagtagatatcaatatatcatgttaacgagattataaagaacagtagtttcagttttatatcaacaatgtaaaccttcttgatggaaccgattaagaactgcatgatgttgcagaaagacactttc

SEQ ID NO: 13 - DNA, 551 bp, Gossypium hirsutum
  misc_feature (105)..(105): n is a, c, g, or t
cattgaagcggcagctacaatgatggcggtggagaacttcttcatgtccatggcgtataaagggattattaaacgcaccggaaatgaaagagacctgcgtttaantactggtgaatgggaagccgccggtgattattatttgctttgttattgcaggtctgaagaacaatgatggtgggggaggtgatttataggtgaaaattaatggctatttttagtgatgacattcaaaaggataattacgtgtttggtttttgcatggtttgcgggatagacatgcatttttaaaaacgattttgacagggttttacgtaagggattttaaattttaagattatatgataatgatttttttatttaaatttaattaaattatgtattttcacttttttttaaatcacaaaactttaattagagttggatatattaattttgataaattattttatggaccctttctattccattacataatcccgtccaataaaaaagggacacatcatttttaatttgaaaatttaatccaaatgtacatataatgcctgtctctt

SEQ ID NO: 14 - DNA, 631 bp, Gossypium hirsutum
  misc_feature (354)..(354): n is a, c, g, or t
atgttaattgccataggctcaaaatgtgctttgcatgctgggtttcccataacattgcactgagttatgaattttttatggctaattgcatgttaaggattgatgaaattttgtacaaaatattaaatcaaaacagataaaaacctttctatctgtaatgcatgttttcatctgcacagcaggcatgtgcacagatcacaaaccatcacaacataaacctttggtttgctattatctatgcatttcccgactgttcatttttcttgaagtatttgtcttgcgattatgaatatatgacccttcaatacatggaaaaacttagaaaacaattaaacatacccgtatctgaaactngcccaaatccaagtaacatgggctcgaatatctcttattatattggttacttcccgttggtcttcgtaactctttcaagatatgcttttacttatgagattatgttgttttagttgcttttcttagattttgatccaacattaaatggatgttttatgtgtttcaagggtgctttttgagaaactgaatttaaattacgaggagggtgagagatggattgtgaatctcatccgaaactctaaacttgatgcaaagattgattcaaagactggaaccg

SEQ ID NO: 15 - DNA, 443 bp, Gossypium hirsutum
  misc_feature (160)..(160): n is a, c, g, or t
cactatacacgcgcacacatatatatagggttcgaattcggtacacctagcaatacaatcttttaactccacaaaggagttaaaaaaactgccacatgtcctatttttattattattttactacaccttaagggtatgtattacctattcaaccttgctnatggagactaaacaagccactagcagaattttccatggatacacacacaggccagtccaataaagagtaacatactgtggcaatatatgacnaatncacgagcgcgcatatatatcataatgacatatttgtcatactatggaagattctttttcttttttaaatnacaaattagattatactgggcaacttaaaccgtcattcaggctcaccaattaacaaccaataaaatgctaaaaccaacactatatgtatatctgagaaaatgcatacacatataaat

SEQ ID NO: 16 - DNA, 481 bp, Gossypium hirsutum
  misc_feature (152)..(152): n is a, c, g, or t
gggaatatgcgtgttttatatatcaaaatcggacaatttgttctgccaggaaagaacttttcattttatcaaagtaatttgaataagtctatcaaatttcaaataagacattaagatgaaagaatgaaaatgttaaccttaagatataaacnctttgctggaatggaatgaataaatacatcagtnagagcctgaagaatctccggagcacatgatgataatgccttgatatttttggttgcagttttctttgaataagtgnctggaatnctgagctctaatttagagtctcccattacagtattgtttgattcatcagcatcctctccagatctaagtatatttttgttttggttgacaagaatctacagaaaaaataacaccactatagatattatacaagagaaaagaaaacaacnaacaaaagttatttggaaagaggatcaacgtaaggttctaggacttacaaaactaatggtaa

SEQ ID NO: 17 - DNA, 581 bp, Gossypium hirsutum
  misc_feature (208)..(208): n is a, c, g, or t
gtttgattcagttgaaggttcgagagataagataaccacctggttcaaagaagtaaatcacaatttaggcaaaaatgtaatacaaagtacttacatcataacaaaaaaggatgaaatattatactttgctcgtacgaaagcctatctcatcaaaaatgtcattttcaagctgcaagatattgtctcccagaaaagaaaccggcttctnaactttaaaacttgctactatatcatgatctggacaataaaataaccaaatgttanatcacttccataattataaattcaatattggtccaaccccacagcaaatatngaattgccaatcctacggaccagcttcgaccagaaaaccgtatataaataactataacaattattatatgaatgcagagattaataaccaagttgatacatccaaaaaaattctcaaaacaataccgatgacaagtacttatatgcaatatacgtgatataacatccaatagtaattgatctatgaaatgttgaacaattcggtcagaaaaccgtataaacaacaacaatggttatatgaatgcagagatcaataaccaagttaa

SEQ ID NO: 18 - DNA, 652 bp, Gossypium hirsutum
  misc_feature (221)..(221): n is a, c, g, or t
atgaatgctgacgacttgctcgatgatttctctaccgaaactttgcggaaagatctaatggctgggaacaagctgatgaaagaggtacgccttttcttttcaagctcaaatcactttgcttacggtctcaaaatgggtcagaaaattaaggccattaaggcgaggttagcttcaattgaaagtgaggccaacacttttggctgcatggtgcgtgaccgccnagtggaaacctctttcatgattaaaaagagacagcgaacacactcttttgtgagtaaagataaaataatagggagggatgatgataaagcggctcttttaaaactcatgttagagtttgaaagtgaagagaacgtttacatcattccagttgtggagtttggaaggttagggaagactgcattggcgcagtttgtttataatgataaaatggtctatgattattttcaattgaggatgtgggtgtgtgtttcagatgtttttgatgtcaaattaattttagaaaacattattaaatctataactggccaagtaccagatcaaaatctcgaaattgaccaattgcaaaaacaacttcgagataaaattggtggaaaaaaatatttgcttgttttggatgacatttagaatgaagagagggaagaatgcgtta

SEQ ID NO: 19 - DNA, 579 bp, Gossypium hirsutum
  misc_feature (356)..(356): n is a, c, g, or t
tgcatcacagaacattgaattttagggttgagagaagaaagagtaaaaaccaacaacgattcctcaacccatgtctttgccttcgcctcctatcgccattctctctacacctttaagtcgcactttcaggagggagagatctgtcattgtttggcatccggagagtgttttcttccactttctgagacgtgcattatattactacagtagtcttgatcgtgaaattcaggtaagttcttggtattgttttgttgagtctaatttgttatggttttcgttgatcgaatcagttatagaccatcagatttgggattcaacaaagtacaagtaggacgtgcgcaatggacattgacacngagtttgagggtctgaatttgttgtttaagtcttaaactagtaatggattttccttttgcctgagttctttgttaacttaattattgtaaaattgatgtaaacttatatgagaagttgctgctgttagctctttccataagcattttccataagcatcggaagcttaaaaggttacttttaacctgtttatcatcttttcttttggactgtaattgtatgata

SEQ ID NO: 20 - DNA, 620 bp, Gossypium hirsutum
  misc_feature (409)..(409): n is a, c, g, or t
tcaaagttccatccaacatagaagttcaagcctgaaagttccaccattaacgatttcttcttgtcaaccccatcttcaacatcctgcaaatgcatctgttccattgagctcaattctatgttggaagcaccattggctactggacttccattaatttcaagtccagacgtgtctacacttccactcaatgtctccatcccaacactgttattttctcaattttccaacctttgtctaaagcaaatttaccctcctgtttgaaaactgacgatcttcatcggttgattgggcttttgaaaaatatatctttttcttttttggtacctttcaaagtccattcaagcagcaaagcaaacaagcaacttattcacagatattggatcattacgcatgataacgggatttattngtgttcatcggattgatcatttagcccaatagactttgagcctaaacccaagatctgtcctgaattaaaagaaataaaaattggatttcaatttccaaaattaaaaaacaactgaaattataacatcaaatttgcattactgccaccaaggcaccaatcaagatttgaacccttcttcttttgacatggagaaggatgactgttgcagtta

SEQ ID NO: 21 - DNA, 410 bp, Gossypium hirsutum
  misc_feature (338)..(338): n is a, c, g, or t
ttcaacaagctgtactttaagctacacgtctaaatgtttgtgttttaccaagctgttcatctaattcttggcacctctaggtgcagttgctggcacccctttgttcccagatgcttctgcatttgaatggataacttgagcacatgtactatcagggcactcagcaactcgatcctgaggtgagactggactgggggcttttgatttctgctgcttgtttcgtccaagaactctgcaaaaggagaatccgttgataggttccagtatttgtaacaactttcataaacttctaatcccatggtctgagagaaattcaattcaaccatcaaagatgacancattcgaggtaaaagtgcaaataccatcagataatgtaaccaatgatttgaaatggatacaatctgtctctt

SEQ ID NO: 22 - DNA, 749 bp, Gossypium hirsutum
  misc_feature (139)..(139): n is a, c, g, or t
cagatacaaagcaatagcgaagaaatgggagacggtccgagtcgaggaacttgaaatcgaggaggatgagttcgttgttgttaactgtttgtatcgtgctaagaatttgcttgatgaaaccgtggcggtacatagtccnaggaatcttgttctcaacttgatacggaagattaatccgaatttgttcatccatgggattattaatggtgcttacaatgctccattctttgtaacacggttccgagaggccttgtttcatttctcatcaatgtttgacatgctcgacgcgatcgtgcctcgcgaagattgggaaaggatgttgatcgagagagagatcttaggcagggaggccttgaatgccattgcttgtgagagttgggagagagtagagcggccggaaacagtcaaacaatggcacgcacgtatcctaagggccggttttctacaacagccattcgaacgcgagatagtcaaggaagcattcgagcgagtccagacgttttaccacaaggatttcgtgatcgacgaagataaccggtggctggtacaagggtggaaaggcagaataatctatgccctttctgcttggaaacctgataaggatatcgaaaatttaggttgcgttccacccggttccagaactcgttcgttactgtacatatcaacggaggcacacaacaaagccacgagggtcggtaggggactaaagagacaatactttggatgtctcgagatatacgtctcggaac

SEQ ID NO: 23 - DNA, 516 bp, Gossypium hirsutum
  misc_feature (51)..(52): n is a, c, g, or t
gatttagcttgtccagtctgctgcaaaatgaaatgtcggagcaatgtaganncgaaaacatataaaaaggagaggaaaaagaatgaaaaaacaacctgttggttactttgatcttttgaggaagtcactgtttgattctgttgttgcaattgcggcacntatgtaacggaatgagaacctccaanagtgttgccgttcatctgaccacggtttggcctggtttgtcttggagcagtagggaactatcacatggtatagtaagtactcatattcgtacagttcgaaccgctaacgtacttgtatataaatacacataccaggggaagtgatgagtattgaaagactggtctggttggtggtgcaaagccattagctctccatcctggccttaaacctaaaggttggtgcatcatcccgggccttaggggtacttgtgaaacaaagccagttggagatgcgtagtaaagcggagggtatcctcccgggagaacaactgttgaaggccctgctaatcct

SEQ ID NO: 24 - DNA, 453 bp, Gossypium hirsutum
  misc_feature (318)..(318): n is a, c, g, or t
gatagacaattacatttaaaatggaaataagataaaacaataatttaaaacaattcaaaactagaataaaaataataactttatttctaagattacgttattatgacccctagattgatggttggtagagcagtaatcttagattgacaattatgaaaaaagtgtgaacagttgatggtaaaagaaaggtggattttccaacttaggaagttgaaatgatgaactcttgtagtgagttacggtagcatagaaactgaatgcaaaatgtgatttgacatctattaggcaaggcccctcaaccgactaccaagataatcnggcacgaacctcactgctttgaactaatagatagaagccgatgattggttgntaataaagaaaaccggttggttggctttggtgaataagagttggtgttgtttcagccaaaaaggtaaatagttagaactatac

SEQ ID NO: 25 - DNA, 450 bp, Gossypium hirsutum; identical to SEQ ID NO: 11
  misc_feature (122)..(122): n is a, c, g, or t

SEQ ID NO: 26 - DNA, 548 bp, Gossypium hirsutum
  misc_feature (449)..(449): n is a, c, g, or t
tgcgtgataaattatatccattttccttgaaagataaatatggtggctgtgaaaccaataatttacggacaacagattgcttagatcataaacgtgatgggcttcatgtgtatttcaatgggcatagccttaaggtgaagaagtgtggtgttagaatagtgtatgagaaagatttggaagaaataaaagagttgcagtgccatacccctcaatcttcaccaaattttgaacacatccaccaacactctgctcacaacgatggatcagtaggtagcacttctgacattaaacaagaacgtaatatctccgaggaagcggaggaagaggggcagcaaccaaaactgttgcaaaaaattttcaattttataatgggccaatcagggaagaagcattaactgtggtaaactacttaaccaatcttgtcctattaactttttttcacatctttnatttaatgtgatcaatctagacttacttacgatccttcttacataccacaaagttataaatcttttactcatattcaacaggagctcatattccgtaaa

SEQ ID NO: 27 - DNA, 415 bp, Gossypium hirsutum
  misc_feature (209)..(209): n is a, c, g, or t
gcccttgattcggtttgtattatcttctaatccaatctatttgctatctccgaagtttgttgggagaaaagcttgaccgtattatgcgaacgttttggtggggtcatgatccgaatcagaggaaacttcactttgggaaaccgaaaaccaatggtggacttagtattcacagcatggagtgatgcattacttagtaagcaggcctggangttactgactgaaccccaacacttcgccatataggaaaatatcatcgacaccaacatttctttaatggaagagtttagttgaaggagacgagatggttgtttgagtggtattgatatctaaatttgcaaggtgcaaacaactctttttaagtgactaggtgatggatgatgggtagacaaactttctaacccacgcttctcacact

SEQ ID NO: 28 - DNA, 631 bp, Gossypium hirsutum; identical to SEQ ID NO: 14
  misc_feature (354)..(354): n is a, c, g, or t

SEQ ID NO: 29 - DNA, 652 bp, Gossypium hirsutum; identical to SEQ ID NO: 18
  misc_feature (221)..(221): n is a, c, g, or t

SEQ ID NO: 30 - DNA, 579 bp, Gossypium hirsutum; identical to SEQ ID NO: 19
  misc_feature (356)..(356): n is a, c, g, or t

SEQ ID NO: 31 - DNA, 585 bp, Gossypium hirsutum
  misc_feature (166)..(166): n is a, c, g, or t
tacctcccagttacaaaagtcccaactttccatctcaaccgtccatcaatcttgatcatcaaaaaaacactcccggccatctattcagcacccaatgcgacggaatattccggtgagatcggtaccatagtaccatatattataggtgaccaaatattgacctcantatggccttgtccagagtgttatttcttggttcccgttagaagcgtaaacgtcgagccggtcgtaataaacaccgatatcgtcgtttgggttacgtgaacggactgtgatttgaaagtttgaagtgagggagttgacggtggtggcgttgaaggcgtagacggtggtgtcgaggagagtgaagttggatttgctgggaggaaggattgcccatatgagtaagattgtgatgagaatgaggaggattagaatacaagcgatgactcggcgaaaaaatttctggcgggatttgtggtggtggccgccgcagtcttagctaccagacatggtggatttggaagttattgggtttctttttggtgattgtgtgaggatttagtgatggaatagtattcagtgtgtgctattagctttcttgtg

SEQ ID NO: 32 - DNA, 592 bp, Gossypium hirsutum
  misc_feature (219)..(219): n is a, c, g, or t
acagaaatagaaatgattgcagtctgtctattttctttccttttattggaattaaatattgatgttttttctcgaaataaagaaatttatgcatgtcatatgggctttgatatcttctcattgttgagaacttggttctgaaatggttactatgttctaattgttttttttttctgatttttagggaaaacacgggcaagagatgctgcgctaaatgcnatccagtcgcctttgttagatcttggtatagaaagggctactggaattgtttggaacataactggtggaagtcatttaaccttgtttaaggtaacgcgccatctccgactctctcagtgtgtctgcgttttcttgcggagactttatatattatcttgatattgagcgcaaaattgttgattattcgtaaatggagcacttccctgtaggagaagagtcattgaagcccaagcggccccaccatatctgatacctgcattcttgagtaacagacaagtgaataattggaaatagtatagtaatgaatactactttgtcattgaaatgttatacatgggaaaccttgattatattgactgatccacttgggttc

SEQ ID NO: 33 - DNA, 620 bp, Gossypium hirsutum; identical to SEQ ID NO: 20
  misc_feature (409)..(409): n is a, c, g, or t

SEQ ID NO: 34 - DNA, 574 bp, Gossypium hirsutum
  misc_feature (255)..(255): n is a, c, g, or t
ctttactattcgcggtggctgcctcttttcttctctacacgtgagcagtactgaaatttgcagactttcttttcttcatttcctagtcgtttcgagtttctttagtttataattttctatcagttgtttgaagaaaacatttcggtgttgttgatttgcttgctctttatgttttttatttgattattaaacgactcatcatctgatctgagttaaaattttcagatctgtacagttttttttgaatagtctccnagcaactatgggccatcaacagcttgcgagggaggcatcttaagcacaatatgactaaacgaacctccattgttggcaggaagcaagaaaagaagtcgggagagagttttcgatcggttccctttagcttctcaaaacttatctgaaattctttcccggcttcttttcttgctttcgttttattgttcgtttgcgttgtgcttcagatgtcgcacatccaggctgtttgaagttgtttgtaggccaacctaatctccatggaagaaatccagtagccgaagttgaggtgaagtggagattgttcgaaaaaacactataa

SEQ ID NO: 35 - DNA, 687 bp, Gossypium hirsutum
  misc_feature (525)..(525): n is a, c, g, or t
taaggctggtcaagacacattgatttcagtatataatatacatcattcctcacaggtgcttgcttattttcatcatactcgtaattttttttctttgaattttttcctggttattactcctatgttgttccgtttcttcatttttcttaaaatgcctgtgtttgatacctttgtccgatgtatataacctaaaagaccccgccaaatatatgggaatacttagaaaaaattttgaaaatatccaaatcctctgtgaaatacctcattttctgttgttcgcagcctacgtatcaaattgtacatgcactatctctatgctgaatttgctgactgcataaccgatcaacgtttggtttccttcaaatagaatattctaagtttgaaaatagggagttttacgattcctaccatttgttttcttggacagcaataagcgatattccgtctttttcttctttccaggtctgggaaagagctgaagagtttgtgcccgagaggttcgacttggaaagctcagtccctaangaatcaaatacagattacaggtacgaaaaacaaccgtgttttactagttttctccctgtccctgatatccttcccacatttgcattacatactcttcatcatattaatacggtagaacatcttcaggttcattccgttcagcgggggtcctcgtaaatgtgt

SEQ ID NO: 36 - DNA, 25 bp, Gossypium hirsutum
ccaatgcaagtgacccaattctaaa

SEQ ID NO: 37 - DNA, 25 bp, Gossypium hirsutum
catgactataactgcaccaggctat

SEQ ID NO: 38 - DNA, 25 bp, Gossypium hirsutum
cccactctctcttaacaacatcaca

SEQ ID NO: 39 - DNA, 27 bp, Gossypium hirsutum
gaacaaccatctgttgaaatcttttgc

SEQ ID NO: 40 - DNA, 28 bp, Gossypium hirsutum
tgacacagcattttcttacactgttttg

SEQ ID NO: 41 - DNA, 32 bp, Gossypium hirsutum
tcaaccagtagatctccaataaacttaaatgg

SEQ ID NO: 42 - DNA, 25 bp, Gossypium hirsutum
ggtggtggcagataaagatattgca

SEQ ID NO: 43 - DNA, 27 bp, Gossypium hirsutum
gagtccttgtaggatcatatccccata

SEQ ID NO: 44 - DNA, 24 bp, Gossypium hirsutum
ttacgaaccacctttgaactgtga

SEQ ID NO: 45 - DNA, 27 bp, Gossypium hirsutum
agccgttgaataaatgcatgcttaaag

SEQ ID NO: 46 - DNA, 22 bp, Gossypium hirsutum
ccaagatactggctgggaagag

SEQ ID NO: 47 - DNA, 20 bp, Gossypium hirsutum
gcccctgacgcatgttctag

SEQ ID NO: 48 - DNA, 21 bp, Gossypium hirsutum
acgcaccggaaatgaaagaga

SEQ ID NO: 49 - DNA, 31 bp, Gossypium hirsutum
cccttcaatacatggaaaaacttagaaaaca

SEQ ID NO: 50 - DNA, 33 bp, Gossypium hirsutum
ctacaccttaagggtatgtattacctattcaac

SEQ ID NO: 51 - DNA, 38 bp, Gossypium hirsutum
gacattaagatgaaagaatgaaaatgttaaccttaaga

SEQ ID NO: 52 - DNA, 25 bp, Gossypium hirsutum
gctgcaagatattgtctcccagaaa

SEQ ID NO: 53 - DNA, 21 bp, Gossypium hirsutum
caacacttttggctgcatggt

SEQ ID NO: 54 - DNA, 20 bp, Gossypium hirsutum
caagtaggacgtgcgcaatg

SEQ ID NO: 55 - DNA, 30 bp, Gossypium hirsutum
caaacaagcaacttattcacagatattgga

SEQ ID NO: 56 - DNA, 26 bp, Gossypium hirsutum
aacttctaatcccatggtctgagaga

SEQ ID NO: 57 - DNA, 27 bp, Gossypium hirsutum
cgtgctaagaatttgcttgatgaaacc

SEQ ID NO: 58 - DNA, 25 bp, Gossypium hirsutum
gtcactgtttgattctgttgttgca

SEQ ID NO: 59 - DNA, 17 bp, Gossypium hirsutum
aggcaaggcccctcaac

SEQ ID NO: 60 - DNA, 22 bp, Gossypium hirsutum
ccaagatactggctgggaagag

SEQ ID NO: 61 - DNA, 30 bp, Gossypium hirsutum
gtggtaaactacttaaccaatcttgtccta

SEQ ID NO: 62 - DNA, 25 bp, Gossypium hirsutum
acagcatggagtgatgcattactta

SEQ ID NO: 63 - DNA, 31 bp, Gossypium hirsutum; identical to SEQ ID NO: 49

SEQ ID NO: 64 - DNA, 21 bp, Gossypium hirsutum
caacacttttggctgcatggt

SEQ ID NO: 65 - DNA, 20 bp, Gossypium hirsutum
caagtaggacgtgcgcaatg

SEQ ID NO: 66 - DNA, 25 bp, Gossypium hirsutum
ggtgagatcggtaccatagtaccat

SEQ ID NO: 67 - DNA, 19 bp, Gossypium hirsutum
aacacgggcaagagatgct

SEQ ID NO: 68 - DNA, 30 bp, Gossypium hirsutum; identical to SEQ ID NO: 55

SEQ ID NO: 69 - DNA, 31 bp, Gossypium hirsutum
aacgactcatcatctgatctgagttaaaatt

SEQ ID NO: 70 - DNA, 22 bp, Gossypium hirsutum
gagaggttcgacttggaaagct

SEQ ID NO: 71 - DNA, 18 bp, Gossypium hirsutum
ccttgctgctcggagctt

SEQ ID NO: 72 - DNA, 28 bp, Gossypium hirsutum
gcatgctcttgttggatatatacccata

SEQ ID NO: 73 - DNA, 31 bp, Gossypium hirsutum
gaccacatgcatagcataactttttaattca

SEQ ID NO: 74 - DNA, 24 bp, Gossypium hirsutum
tgatgagaacttgcggaaaatcca

SEQ ID NO: 75 - DNA, 32 bp, Gossypium hirsutum
aaggctactcgggaaaaagaataacataaata

SEQ ID NO: 76 - DNA, 27 bp, Gossypium hirsutum
gtccgaatttgaaattggacaaggaaa

SEQ ID NO: 77 - DNA, 25 bp, Gossypium hirsutum
cactttggagataccctttcaggaa

SEQ ID NO: 78 - DNA, 36 bp, Gossypium hirsutum
ggtttagttcattggaaactatattactctgattct

SEQ ID NO: 79 - DNA, 21 bp, Gossypium hirsutum
tcacaatgccgccgctaataa

SEQ ID NO: 80 - DNA, 21 bp, Gossypium hirsutum
tgcaactggcactcaggtaag

SEQ ID NO: 81 - DNA, 22 bp, Gossypium hirsutum
gtctggccatggattagccttt

SEQ ID NO: 82 - DNA, 24 bp, Gossypium hirsutum
gccattcatgtatcgcatcatctt

SEQ ID NO: 83 - DNA, 14 bp, Gossypium hirsutum
ccggcggcttccca

SEQ ID NO: 84 - DNA, 25 bp, Gossypium hirsutum
gatattcgagcccatgttacttgga

SEQ ID NO: 85 - DNA, 25 bp, Gossypium hirsutum
gcctgtgtgtgtatccatggaaaat

SEQ ID NO: 86 - DNA, 22 bp, Gossypium hirsutum
catgtgctccggagattcttca

SEQ ID NO: 87 - DNA, 33 bp, Gossypium hirsutum
taacatttggttattttattgtccagatcatga

SEQ ID NO: 88 - DNA, 33 bp, Gossypium hirsutum
catccctccctattattttatctttactcacaa

SEQ ID NO: 89 - DNA, 32 bp, Gossypium hirsutum
gcaaaaggaaaatccattactagtttaagact

SEQ ID NO: 90 - DNA, 25 bp, Gossypium hirsutum
cagatcttgggtttaggctcaaagt

SEQ ID NO: 91 - DNA, 30 bp, Gossypium hirsutum
tggttacattatctgatggtatttgcactt

SEQ ID NO: 92 - DNA, 28 bp, Gossypium hirsutum
cggattaatcttccgtatcaagttgaga

SEQ ID NO: 93 - DNA, 21 bp, Gossypium hirsutum
ggtcagatgaacggcaacact

SEQ ID NO: 94 - DNA, 29 bp, Gossypium hirsutum
ccaatcatcggcttctatctattagttca

SEQ ID NO: 95 - DNA, 22 bp, Gossypium hirsutum
gtctggccatggattagccttt

SEQ ID NO: 96 - DNA, 30 bp, Gossypium hirsutum
aactttgtggtatgtaagaaggatcgtaag

SEQ ID NO: 97 - DNA, 23 bp, Gossypium hirsutum
ttcctatatggcgaagtgttggg

SEQ ID NO: 98 - DNA, 25 bp, Gossypium hirsutum
gatattcgagcccatgttacttgga

SEQ ID NO: 99 - DNA, 33 bp, Gossypium hirsutum; identical to SEQ ID NO: 88

SEQ ID NO: 100 - DNA, 32 bp, Gossypium hirsutum; identical to SEQ ID NO: 89

SEQ ID NO: 101 - DNA, 21 bp, Gossypium hirsutum
cgcttctaacgggaaccaaga

SEQ ID NO: 102 - DNA, 26 bp, Gossypium hirsutum
tccagtagccctttctataccaagat

SEQ ID NO: 103 - DNA, 25 bp, Gossypium hirsutum
cagatcttgggtttaggctcaaagt

SEQ ID NO: 104 - DNA, 21 bp, Gossypium hirsutum
caagctgttgatggcccatag

SEQ ID NO: 105 - DNA, 27 bp, Gossypium hirsutum
gggagaaaactagtaaaacacggttgt

SEQ ID NO: 106 - DNA, 20 bp, Gossypium hirsutum
ttgaaaacataaaaagacca

SEQ ID NO: 107 - DNA, 18 bp, Gossypium hirsutum
actcgggaatgagtatag

SEQ ID NO: 108 - DNA, 20 bp, Gossypium hirsutum
agattggtgtcttatgtgtg

SEQ ID NO: 109 - DNA, 15 bp, Gossypium hirsutum
cagttgggcagcaaa

SEQ ID NO: 110 - DNA, 21 bp, Gossypium hirsutum
ctttttatttttcgtaaggcc

SEQ ID NO: 111 - DNA, 16 bp, Gossypium hirsutum
ttgagtcgacattatc

SEQ ID NO: 112 - DNA, 16 bp, Gossypium hirsutum
ttgtcaccgctcctac

SEQ ID NO: 113 - DNA, 18 bp, Gossypium hirsutum
cgtgttttgatgcttatc

SEQ ID NO: 114 - DNA, 18 bp, Gossypium hirsutum
tccgtattcaactaaaca

SEQ ID NO: 115 - DNA, 16 bp, Gossypium hirsutum
cacatgcaggtttgtg

SEQ ID NO: 116 - DNA, 20 bp, Gossypium hirsutum
ctattattaggcaagattaa

SEQ ID NO: 117 - DNA, 17 bp, Gossypium hirsutum
catacatgttgaagctt

SEQ ID NO: 118 - DNA, 17 bp, Gossypium hirsutum
ttcaccagtaattaaac

SEQ ID NO: 119 - DNA, 14 bp, Gossypium hirsutum
tttgggcgagtttc

SEQ ID NO: 120 - DNA, 21 bp, Gossypium hirsutum
tgtttagtctccatcagcaag

SEQ ID NO: 121 - DNA, 15 bp, Gossypium hirsutum
cagcaaagcgtttat

SEQ ID NO: 122 - DNA, 17 bp, Gossypium hirsutum
ccggcttcttaacttta

SEQ ID NO: 123 - DNA, 14 bp, Gossypium hirsutum
cactgggcggtcac

SEQ ID NO: 124 - DNA, 16 bp, Gossypium hirsutum
attgacacagagtttg

SEQ ID NO: 125 - DNA, 18 bp, Gossypium hirsutum
cgatgaacactaataaat

SEQ ID NO: 126 - DNA, 16 bp, Gossypium hirsutum
aagatgacaacattcg

SEQ ID NO: 127 - DNA, 16 bp, Gossypium hirsutum
catagtccgaggaatc

SEQ ID NO: 128 - DNA, 16 bp, Gossypium hirsutum
ttgcggcacgtatgta

SEQ ID NO: 129 - DNA, 21 bp, Gossypium hirsutum
ctaccaagataatcaggcacg

SEQ ID NO: 130 - DNA, 20 bp, Gossypium hirsutum
ctattattaggcaagattaa

SEQ ID NO: 131 - DNA, 20 bp, Gossypium hirsutum
atcacattaaataaaagatg

SEQ ID NO: 132 - DNA, 18 bp, Gossypium hirsutum
cctggaagttactgactg

SEQ ID NO: 133 - DNA, 14 bp, Gossypium hirsutum
tttgggcgagtttc

SEQ ID NO: 134 - DNA, 14 bp, Gossypium hirsutum
cactgggcggtcac

SEQ ID NO: 135 - DNA, 16 bp, Gossypium hirsutum
attgacacagagtttg

SEQ ID NO: 136 - DNA, 17 bp, Gossypium hirsutum
aaggccatattgaggtc

SEQ ID NO: 137 - DNA, 16 bp, Gossypium hirsutum
actggatagcatttag

SEQ ID NO: 138 - DNA, 16 bp, Gossypium hirsutum
atgaacacaaataaat

SEQ ID NO: 139 - DNA, 20 bp, Gossypium hirsutum
tttgaatagtctccaagcaa

SEQ ID NO: 140 - DNA, 15 bp, Gossypium hirsutum
tccctaacgaatcaa

SEQ ID NO: 141 - DNA, 21 bp, Gossypium hirsutum
cttgaaaacataagaagacca

SEQ ID NO: 142 - DNA, 18 bp, Gossypium hirsutum
actcgggaataagtatag

SEQ ID NO: 143 - DNA, 20 bp, Gossypium hirsutum
agattggtgtcttgtgtgtg

SEQ ID NO: 144 - DNA, 15 bp, Gossypium hirsutum
cagttgggcggcaaa

SEQ ID NO: 145 - DNA, 21 bp, Gossypium hirsutum
ctttttatttttcataaggcc

SEQ ID NO: 146 - DNA, 18 bp, Gossypium hirsutum
ctttgagttgacattatc

SEQ ID NO: 147 - DNA, 19 bp, Gossypium hirsutum
attttgtcaccactcctac

SEQ ID NO: 148 - DNA, 19 bp, Gossypium hirsutum
ccgtgttttgattcttatc

SEQ ID NO: 149 - DNA, 20 bp, Gossypium hirsutum
tttccgtattcaattaaaca

SEQ ID NO: 150 - DNA, 16 bp, Gossypium hirsutum
cacatgcagttttgtg

SEQ ID NO: 151 - DNA, 17 bp, Gossypium hirsutum
ttattaggcgagattaa

SEQ ID NO: 152 - DNA, 17 bp, Gossypium hirsutum
catacatgttcaagctt

SEQ ID NO: 153 - DNA, 17 bp, Gossypium hirsutum
ttcaccagtatttaaac

SEQ ID NO: 154 - DNA, 13 bp, Gossypium hirsutum
ttgggcaagtttc

SEQ ID NO: 155 - DNA, 21 bp, Gossypium hirsutum
tgtttagtctccatgagcaag

SEQ ID NO: 156 - DNA, 16 bp, Gossypium hirsutum
ccagcaaagagtttat

SEQ ID NO: 157 - DNA, 16 bp, Gossypium hirsutum
cggcttctcaacttta

SEQ ID NO: 158 - DNA, 15 bp, Gossypium hirsutum
ccactaggcggtcac

SEQ ID NO: 159 - DNA, 15 bp, Gossypium hirsutum
ttgacacggagtttg

SEQ ID NO: 160 - DNA, 16 bp, Gossypium hirsutum
atgaacacaaataaat

SEQ ID NO: 161 - DNA, 16 bp, Gossypium hirsutum
aagatgacagcattcg

SEQ ID NO: 162 - DNA, 16 bp, Gossypium hirsutum
catagtccaaggaatc

SEQ ID NO: 163 - DNA, 16 bp, Gossypium hirsutum
ttgcggcacatatgta

SEQ ID NO: 164 - DNA, 18 bp, Gossypium hirsutum
ccaagataatcgggcacg

SEQ ID NO: 165 - DNA, 17 bp, Gossypium hirsutum
ttattaggcgagattaa

SEQ ID NO: 166 - DNA, 19 bp, Gossypium hirsutum
tcacattaaatgaaagatg

SEQ ID NO: 167 - DNA, 17 bp, Gossypium hirsutum
ctggaggttactgactg

SEQ ID NO: 168 - DNA, 13 bp, Gossypium hirsutum
ttgggcaagtttc

SEQ ID NO: 169 - DNA, 15 bp, Gossypium hirsutum
ccactaggcggtcac

SEQ ID NO: 170 - DNA, 15 bp, Gossypium hirsutum
ttgacacggagtttg

SEQ ID NO: 171 - DNA, 17 bp, Gossypium hirsutum
aaggccataatgaggtc

SEQ ID NO: 172 - DNA, 15 bp, Gossypium hirsutum
ctggatggcatttag

SEQ ID NO: 173 - DNA, 18 bp, Gossypium hirsutum
cgatgaacactaataaat

SEQ ID NO: 174 - DNA, 22 bp, Gossypium hirsutum
tttttgaatagtctccgagcaa

SEQ ID NO: 175 - DNA, 15 bp, Gossypium hirsutum
tccctaaggaatcaa